(19) United States
(12) Patent Application Publication
(10) Pub. No.: US 2007/0066544 A1
(43) Pub. Date: Mar. 22, 2007

(54) 3-Decladinosyl 9a-n-carbamoyl and 9a-n-thiocarbamoyl derivatives of 9-deoxo-9-dihydro-9a-aza-9a-homoerythromycin a

(75) Inventors: Zorica Marusic-Istuk, Samobor (HR); Nedjeljko Kujundzic, Zagreb (HR); Stjepan Mutak, Zagreb (HR)

Correspondence Address: DARBY & DARBY P.C., P. O. BOX 5257, NEW YORK, NY, 10150-5257, US

(73) Assignee: GlaxoSmithKline Istrazivacki centar Zagreb d.o.o., Zagreb (HR)

(21) Appl. No.: 10/557,025

(22) PCT Filed: May 11, 2004
(86) PCT No.: PCT/HR04/00014

§ 371 Date: Nov. 13, 2006

(30) Foreign Application Priority Data
May 14, 2003 (HR) P20030381A

(51) Int. Cl. A61K 31/7052; C07H 17/08

(52) U.S. Cl. 514/28; 536/7.4

(57) ABSTRACT

The present invention relates to the new 3-decladinosyl derivatives of 9a-N-carbamoyl- and 9a-N-thiocarbamoyl-9-deoxo-9-dihydro-9a-aza-9a-homoerythromycin A of the general formula (I), 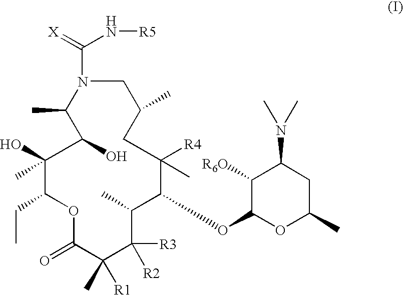 their pharmaceutically acceptable addition salts with inorganic or organic acids and their hydrates, to intermediates for synthesis of other macrolide compounds with antibacterial activity, to the process for their preparation, to their pharmaceutically acceptable addition salts with inorganic or organic acids and their hydrates, to the process for the preparation of pharmaceutical compositions, as well as the use of pharmaceutical compositions for treating bacterial infections.

### CROSS-REFERENCE TO PRIOR APPLICATIONS

This is a U.S. national phase application under 35 U.S.C. §371 of International Patent Application No. PCT/HR2004/000014 filed on May 11, 2004, and claiming priority to Croatian Application P20030381 filed May 14, 2003, both of which are incorporated by reference herein in their entirety. The International Application was published in English on Nov. 25, 2004 as WO 04/101591 A1 under PCT Article 21(2).

### 1) TECHNICAL FIELD OF THE INVENTION

A 61 K 31/70, C 0 7H 17/08

### 2) TECHNICAL PROBLEM

The invention relates to novel compounds from the class of azalide antibiotics. Particularly, the invention relates to novel 3-decladinosyl derivatives from the class of 9a-N-carbamoyl- and 9a-N-thiocarbamoyl-9-deoxo-9-dihydro-9a-aza-9a-homoerythromycin A, to their pharmaceutically acceptable addition salts with inorganic or organic acids, to their hydrates, to the process for their preparation, to the process for preparation of their pharmaceutical compositions and to the use thereof as antibiotics or intermediates for the synthesis of other macrolide antibiotics.

### 3) PRIOR ART

Macrolides are well known agents for treating broad spectrum of infections. Erythromycin A (McGuire J. M., Antibiot. Chemother. 1952; 2: 281) has been for more than 40 years considered as safe and efficient agent for the treatment of respiratory and genital infections caused by Gram-positive and by some Gram-negative bacteria, some species of Legionella, Mycoplasma, Chlamidia and Helicobacter. By oximation of C-9 ketone of erythromycin and subsequent Beckmann rearrangement and reduction, 9-deoxo-9a-aza-9a-homoerythromycin A, the first 15-membered macrolide antibiotics with 9a-amino group incorporated in aglycone ring, is obtained (Kobrehel G. et al., U.S. Pat. No. 4,328,334 May 1982).

By O-methylation of C-6 hydroxyl group of erythromycin clarithromycin is obtained (6-O-metil-erythromycin A) (Morimoto S. et al., J. Antibiotics 1984, 37, 187). In comparison with erythromycin A, clarithromycin is more stable and shows enhanced in vitro activity against Gram-positive strains (Kirst H. A. et al., Antimicrob. Agents and Chemother. 1989, 1419).

9a-N-carbamoyl i 9a-N-thiocarbamoyl derivatives of 9-deoxo-9-dihydro-9a-aza-9a-homoerythromycin A, who shoved significant antibacterial activity, are described. (Kujund\check{z}ić N. et. al., Eur. J. Med. Chem. 1995, 30, 455).

It is known as well that recent research on 14-membered macrolides has lead to the discovery of new classes of macrolide antibiotics, ketolides and anhydrolides. Instead of the neutral sugar L-cladinose known for its instability even in a weakly acidic medium, ketolides possess a keto group on C-3 position (Agouridas C. et al., EP 596802 A1 5/1994, Le Martret O., FR 2697524 A15/94). Anhydrolides are characterized by 2,3-anhydro group (Elliott R. et al., J. Med. Chem. 1998, 41, 1651). Ketolides show a significantly better activity against MLS (macrolide, lincosamide and streptogramin B) induced-resistant organisms (Jamjian C., Antimicrob. Agents Chemother. 1997, 41, 485).

Object of the present invention are 3-decladinosyl derivatives of 9a-N-carbamoyl- and 9a-N-thiocarbamoyl-9-deoxo-9-dihydro-9a-aza-9a-homoerythromycin A, their pharmaceutically acceptable addition salts with inorganic or organic acids, their hydrates, methods and intermediates for their preparation as well as preparation and application methods of pharmaceutical preparations.

### 4) DESCRIPTION OF TECHNICAL PROBLEM WITH EXAMPLES

The invention relates to:
i) novel 3-decladinosyl derivatives of 9a-N-carbamoyl- and 9a-N-thiocarbamoyl-9-deoxo-9-dihydro-9a-aza-9a-homoerythromycin A, their pharmaceutically acceptable addition salts with inorganic or organic acids and their hydrates,
ii) methods for preparation of novel 3-decladinosyl derivatives of 9a-N-carbamoyl- and 9a-N-thiocarbamoyl-9-deoxo-9-dihydro-9a-aza-9a-homoerythromycin A, their pharmaceutically acceptable addition salts with inorganic or organic acids and their hydrates,
iii) use of novel 3-decladinosyl derivatives of 9a-N-carbamoyl- and 9a-N-thiocarbamoyl-9-deoxo-9-dihydro-9a-aza-9a-homoerythromycin A, their pharmaceutically acceptable addition salts with inorganic or organic acids and their hydrates as antibiotics or
iv) use of novel 3-decladinosyl derivatives of 9a-N-carbamoyl- and 9a-N-thiocarbamoyl-9-deoxo-9-dihydro-9a-aza-9a-homoerythromycin A, their pharmaceutically acceptable addition salts with inorganic or organic acids and their hydrates as intermediates for the synthesis of other macrolide antibiotics.

Novel 3-decladinosyl derivatives of 9a-N-carbamoyl- and 9a-N-thiocarbamoyl-9-deoxo-9-dihydro-9a-aza-9a-homoerythromycin A of the general formula (I), 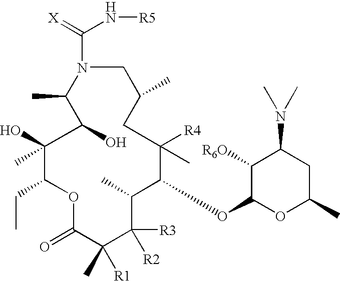
their pharmaceutically acceptable addition salts with inorganic or organic acids and their hydrates, wherein
R₁ individually stands for hydrogen or together with R₂ stands for double bond,
R₂ individually stands for hydrogen, hydroxyl or a group of the formula (II), 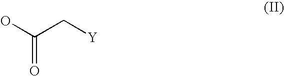
wherein
Y individually stands for monocyclic aromatic ring unsubstituted or substituted with groups which are selected independently from halogen, OH, OMe, NO₂, NH₂
or
R₂ together with R₃ stands for ketone or together with R₁ stands for double bond,
R₃ individually stands for hydrogen or together with R₂ stands for ketone or together with R₄ stands for ether,
R₄ individually stands for hydroxyl, OCH₃ group or together with R₃ stands for ether,
R₅ stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, and m is 0-3,
R₆ individually stands for hydrogen or hydroxyl protected group and
X stands for oxygen or sulphur
are subject of this invention.

Term <<hydroxyl protected group>> includes, but is not limited on benzoyl, benzyloxycarbonyl, acetyl or substituted silyl group in order to block the undesired reaction due the synthesis (Green T. H. and Wuts P. G. M., Protective Groups in Organic Synthesis, 3rd edition, John Wiley & Sons, New York, 1999).

Compounds given by general formula (I), wherein R₁, R₂, R₃, R₄, R₅ R₆ X and Y have the meaning as defined above could be prepared by methods described in this invention. Methods of preparations, which also are subject of this invention, are illustrated by schemes 1. and 2.:

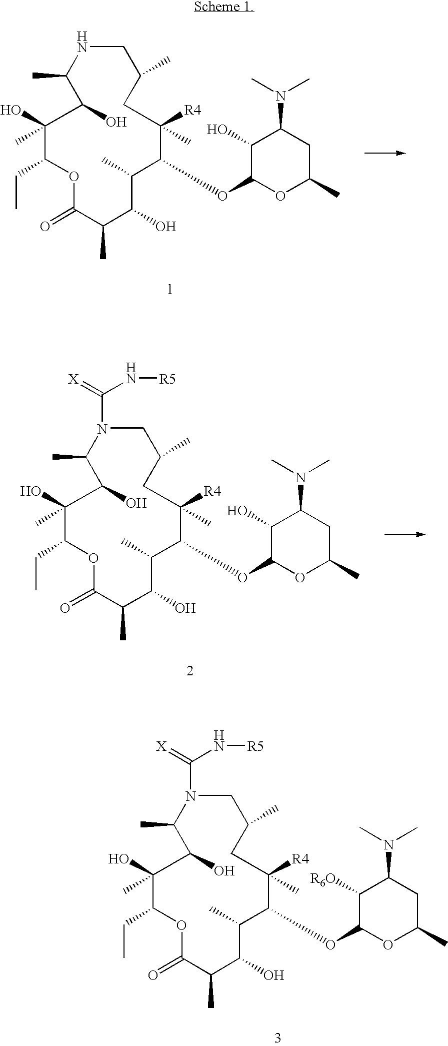

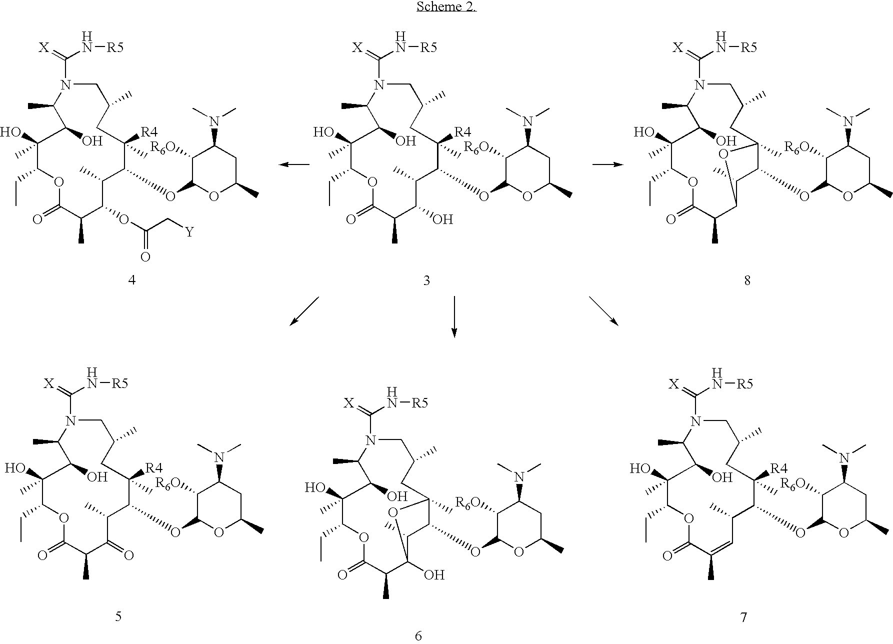

Starting compound described in scheme 1. for synthesis of compounds, which are subject of this invention are prepared by methods described in patent Kobrehel G. et al., U.S. Pat. No. 4,328,334 May 1982 and in the article Denis A. and Agouridas C., Bioorg. Med. Chem. Lett. 1998, 8, 2427 (compound of general formula 1).

Step 1.

Compounds of the formula 1., wherein R₄ individually stands for hydroxyl or OCH₃ group are subjected to a reaction with isocyanate or isothiocyanate of the formula R₅—N═C═X wherein R₅ and X have the above meanings, in a reaction-inert solvent preferably toluene or acetonitril, at a temperature from room temperature to the reflux temperature of the solvent for 30 min to 50 hours, yielding 9a-N-carbamoyl and 9a-N-thiocarbamoyl derivatives of the formula 2. (scheme 1.), wherein R₄ individually stands for hydroxyl or OCH₃ group and R₅ and X have the above meanings.

9a-N-carbamoyl and 9a-N-thiocarbamoyl derivatives of the formula 2 wherein all substituents have the above meanings are subjected to a selective acylation of the hydroxyl group on 2′-position. Acylation is carried out with chlorides or anhydrides of carboxylic acids with up to 4 carbon atoms, preferably with acetic acid anhydrides, in the presence of inorganic or organic base, in a reaction-inert solvent at a temperature from 0-30° C., yielding 2′-O-acyl derivatives of the formula 3 (scheme 1.), wherein R₄ individually stands for hydroxyl or OCH₃ group, R₅ and X have the above meanings and R₆ stands for hydroxyl protected group, preferably acetyl.

As suitable bases sodium hydrogencarbonate, sodium carbonate, potassium carbonate, triethylamine, pyridine, tributylamine are used. As a suitable inert solvent methylene chloride, dichlorethane, acetone, pyridine, ethyl acetate, tetrahydrofuran are used.

Step 2.

2′-O-Acetyl derivatives from the Step 1. are optionally subjected to a reaction with mixed anhydrides of carboxylic acids of the formula Z-COO—R′, wherein Z individually stands for hydrogen or for group Y, which is defined above, R′ stands for the group which is usually used for preparation of mixed anhydrides as pivaloyl-, p-toluensulphonyl-, isobutoxycarbonyl-, etoxycarbonyl- or isopropoxycarbonyl-group, in the presence of inorganic or organic base, in a reaction-inert solvent, preferably methylene chloride at a temperature from 0-30° C. for 3-100 hours yielding compounds of the formula 4. (scheme 2.), wherein R₄ individually stands for hydroxyl or OCH₃ group, R₆ stands for acetyl and substituents R₅, X and Y have the above meanings. Formed compounds are subsequently subjected to deprotection with lower alcohols, preferably in methanol, at a temperature from room temperature to the reflux temperature of the solvent, yielding a compound of the formula 4. (scheme 2.), wherein R₆ stands for hydrogen and R₄, R₅ X and Y have the above meanings,

or optionally

2′-O-acetyl derivatives from the Step 1., wherein R₄ stands for the OCH₃ group and all other substituents have the meanings as in the Step 1. are subjected to oxidation of the hydroxyl group in the C-3 position of an aglycone ring according to a modified Moffat-Pfitzner process with N,N-dimethylaminopropyl-3-ethyl-carbodiimide in the presence of dimethylsulfoxide and pyridinium trifluoracetate as a catalyst in a inert organic solvent, preferably in methylene chloride, at a temperature from 10° C. to room temperature, yielding compounds of the formula 5. (scheme 2.), wherein R₄ stands for the OCH₃ group, R₆ stands for acetyl and supstituents R₅ and X have the above meanings. Formed compounds are subsequently subjected to deprotection with lower alcohols, preferably in methanol, at a temperature from room temperature to the reflux temperature of the solvent, yielding a compound of the formula 5. (scheme 2.), wherein R₆ stands for hydrogen and all other supstituents have the above meanings. Alternatively it is possible to oxidize C-3 hydroxyl group using Dess Martin periodinane reagens

or optionally

2′-O-acetyl derivatives from the Step 1., wherein R₄ stands for hydroxyl and all other supstituents have the meanings as in the Step 1. are subjected to oxidation described to obtain compounds of the formula 5. (scheme 2.), where compounds with 3,6-hemiketal structure given by formula 6. (scheme 2.), wherein R₆ stands for acetyl and R₅ and X have the above meanings are formed. Formed compounds are subsequently subjected to deprotection with lower alcohols, preferably in methanol, at a temperature from room temperature to the reflux temperature of the solvent, yielding a compound of the formula 6. (scheme 2.), wherein R₆ stands for hydrogen and R₅ and X have the above meanings

or optionally

2′-O-acetyl derivatives from the Step 1., wherein R₄ stands for the OCH₃ group and all other substituents have the meanings as in the Step 1., are subjected to the adequate reagents for dehydratation, preferably methylsulfonyl anhydride to transform hydroxyl group on position 3 in good leaving group, in an inert organic solvent, preferably in pyridine, at a temperature from room temperature to the reflux temperature of the solvent for 10-50 hours. Formed intermediate is subsequently subjected to reaction of elimination with adequate reagens, preferably sodium hydride, in a inert organic solvent, preferably in tetrahydrofuran, at a temperature from 10° C. to room temperature, yielding 2,3-anhydro derivatives of the formula 7. (scheme 2.), R₄ stands for the OCH₃ group, R₆ stands for acetyl and R₅ and X have the above meanings. Formed compounds are subsequently subjected to deprotection with lower alcohols, preferably in methanol, at a temperature from room temperature to the reflux temperature of the solvent, yielding a compound of the formula 7. (scheme 2.), wherein R₆ stands for hydrogen and R₄, R₅ and X have the above meanings.

or optionally

2′-O-acetyl derivatives from the Step 1., wherein R₄ stands for the OH group and all other substituents have the meanings as in the Step 1., are subjected to adequate reagents for dehydratation, described to obtain compounds of the formula 7. (scheme 2.), yielding 3,6 cyclic ethers of the formula 8. (scheme 2.), wherein R₆ stands for acetyl and R₅ and X have the above meanings. Formed compounds are subsequently subjected to deprotection with lower alcohols, preferably in methanol, at a temperature from room temperature to the reflux temperature of the solvent, yielding a compound of the formula 8. (scheme 2.), wherein R₆ stands for hydrogen and R₅ and X have the above meanings.

Pharmaceutically acceptable addition salts which are also subject of this invention are prepared by reaction of new compounds of the general formula (I) with at least one eqimolar amounts of suitable inorganic or organic acid as chloride, iodide, sulphate, phosphate, acetic, propionic, trifluoracetic, maleinic, citric, stearic, jantaric, ethyljantaric, mathansulphonic, p-toluensulphonic, laurylsulphonic and other acids in reaction inert solvent. Addition salts are isolated by filtration (if they are insoluble in used solvent), by precipitation, by evaporating the solvent or by liophilisation.

The process is illustrated by the following examples, which do not limit the scope of the invention in any way.

### EXAMPLE 1

### 3-Decladinosyl-9-deoxo-9-dihydro-9a-N—(N′-isopropylcarbamoyl)-9a-aza-9a-homoerythromycin A

To a solution of 3-decladinosyl-9-deoxo-9-dihydro-9a-aza-9a-homoerythromycin A (1.00 g) in toluene (20 ml) isopropylisocyanate (0.17 g) was added. Reaction mixture was stirred for 1 hour at room temperature. The solvent was evaporated and 1.15 g of crude product is obtained.

Crystallization from mixture acetone-petrol ether yielding 0.97 g of chromatographically homogenous title product with following physical-chemical constants:

TLC R_{f}=0.37 (CH₂Cl₂:MeOH=8:2)

IR (KBr) [ν/cm⁻¹]: 3498, 3466, 2974, 2938, 2877, 1726, 1618, 1596, 1528, 1458, 1373, 1279, 1172, 1111, 1082, 1036, 978, 956, 932, 901, 833, 776, 689, 633.

MS m/z: (FAB): MH⁺=662

¹H NMR (300 MHz, CDCl₃) [δ/ppm] 5.03 (13-H), 4.40 (1′-H), 4.37 (9a-NCON′H), 3.92 (1″-H), 3.89 (3-H), 3.50 (11-H), 3.69 (5′-H), 3.63 (5-H), 3.32 (2′-H), 2.60 (2-H), 2.54 (3′-H), 2.28 (3′-N(CH₃)₂), 1.88 (14-Ha), 1.72 (4′-Ha), 1.58 (14-Hb), 1.56 (4-H), 1.34 (4′-Hb), 1.29 (2-CH₃), 1.27 (5′-CH₃), 1.26 (10-CH₃), 1.13 (1″-(CH₃)₂), 1.10 (12-CH₃), 1.06 (8-CH₃), 0.93 (4-CH₃), 0.89 (15-CH₃).

¹³C NMR (75 MHz, CDCl₃) [δ/ppm] 177.3 (1-C), 158.8 (9a-NCONH), 105.8 (1′-C), 95.9 (5-C), 77.7 (13-C), 77.1 (12-C), 74.5 (11-C), 74.9 (3-C), 74.2 (6-C), 73.0 (9-C), 70.3 (5′-C), 69.2 (2′-C), 64.9 (3′-C), 44.6 (2-C), 42.4 (1″-C), 41.7 (7-C), 39.9 (3′-N(CH₃)₂), 38.5 (4-C), 27.6 (4′-C), 25.8 (6-CH₃), 23.1 (1″-(CH₃)₂), 21.2 (14-C), 20.6 (5′-CH₃), 19.4 (8-CH₃), 16.6 (12-CH₃), 15.9 (2-CH₃), 12.2 (10-CH₃), 10.0 (15-CH₃), 7.7 (4-CH₃).

### EXAMPLE 2

### 3-Decladinosyl-9-deoxo-9-dihydro-9a-N—(N′-ethylcarbamoyl)-9a-aza-9a-homoerythromycin A

To a solution of 3-decladinosyl-9-deoxo-9-dihydro-9a-aza-9a-homoerythromycin A (1.00 g) in toluene (20 ml) ethylisocyanate (0.175 g) was added. Reaction mixture was stirred for 1 hour at room temperature. The solvent was evaporated and 1.24 g of crude product is obtained.

Crystallization from mixture acetone-petrol ether yielding 0.98 g of chromatographically homogenous title product with following physical-chemical constants:

TLC R_{f}=0.48 (CH₂Cl₂:MeOH=8:2)

IR (KBr) [ν/cm⁻¹]: 3433, 2975, 2936, 2878, 1730, 1618, 1532, 1458, 1382, 1350, 1267, 1172, 1111, 1077, 1036, 980, 956, 933, 900, 834, 764.

MS m/z: (FAB): MH⁺=648

¹H NMR (300 MHz, CDCl₃) [δ/ppm] 5.03 (13-H), 4.39 (1′-H), 4.56 (9a-NCON′H), 3.86 (3-H), 3.57 (1-H), 3.68 (5′-H), 3.62 (5-H), 3.43 (9-Ha), 3.31 (2′-H), 3.23 (N′CH₂), 2.59 (2-H), 2.53 (3′-H), 2.47 (9-Hb), 2.27 (3′-N(CH₃)₂), 1.90 (14-Ha), 1.71 (4′-Ha), 1.55 (14-Hb), 1.55 (4-H), 1.33 (4′-Hb), 1.28 (2-CH₃), 1.28 (15-CH₃), 1.28 (10-CH₃), 1.27 (5′-CH₃), 1.11 (N′CH₂CH₃), 1.08 (12-CH₃), 1.05 (8-CH₃), 0.92 (4-CH₃), 0.87 (15-CH₃).

¹³C NMR (75 MHz, CDCl₃) [δ/ppm] 177.0 (1-C), 159.5 (9a-NCONH), 105.7 (1′-C), 96.2 (5-C), 77.7 (13-C), 77.1 (12-C), 74.5 (11-C), 75.1 (3-C), 74.1 (6-C), 72.8 (9-C), 70.3 (5′-C), 69.2 (2′-C), 64.9 (3′-C), 44.7 (2-C), 41.1 (7-C), 39.9 [3′N—(CH₃)₂], 38.3 (4-C), 36.5 (N′CH₂), 28.9 (8-C), 27.6 (4′-C), 25.6 (6-CH₃), 21.3 (14-C), 20.6 (5′-CH₃), 19.3 (8-CH₃), 16.6 (12-CH₃), 15.8 (2-CH₃), 15.0 (N′CH₂CH₃), 12.1 (10-CH₃), 10.3 (15-CH₃), 7.6 (4-CH₃).

### EXAMPLE 3

### 3-Decladinosyl-9-deoxo-9-dihydro-9a-N—[N′-(t-buthyl)carbamoyl]-9a-aza-9a-homoerythromycin A

To a solution of 3-decladinosyl-9-deoxo-9-dihydro-9a-aza-9a-homoerythromycin A (1.00 g) in toluene (20 ml) t-buthylisocyanate (0.198 g) was added. Reaction mixture was stirred for 1 hour at room temperature. The solvent was evaporated and 1.20 g of crude product is obtained.

Crystallization from mixture acetone-petrol ether yielding 0.95 g of chromatographically homogenous title product with following physical-chemical constants:

TLC R_{f}=0.42 (CH₂Cl₂:MeOH=8:2)

IR (KBr) [ν/cm⁻¹]: 3497, 2976, 2946, 1725, 1626, 1532, 1456, 1364, 1346, 1317, 1281, 1175, 1112, 1082, 1055, 1034, 977, 956, 931, 901, 865, 776, 685, 634.

MS m/z: (FAB): MH⁺=676

¹H NMR (300 MHz, CDCl₃) [δ/ppm] 5.03 (13-H), 4.52 (9a-NCON′H), 4.41(1′-H), 3.89 (3-H), 3.68 (5′-H), 3.47 (11-H), 3.62 (5-H), 3.41 (9-Ha), 3.32 (2′-H), 2.59 (2-H), 2.54 (3′-H), 2.28 (3′-N(CH₃)₂), 1.88 (14-Ha), 1.72 (4′-Ha), 1.57 (14-Hb), 1.56 (4-H), 1.32 (4′-Hb), 1.31 (N′C(CH₃)₃), 1.30 (2-CH₃), 1.28 (5′-CH₃), 1.24 (10-CH₃), 1.11 (12-CH₃), 1.05 (8-CH₃), 0.92 (4-CH₃), 0.88 (15-CH₃).

¹³C NMR (75 MHz, CDCl₃) [δ/ppm] 177.7 (1-C), 158.7 (9a-NCONH), 105.9 (1′-C), 95.7 (5-C), 77.9 (13-C), 77.1 (12-C), 74.6 (11-C), 74.8 (3-C), 74.3 (6-C), 73.3 (9-C), 70.3 (5′-C), 69.2 (2′-C), 65.0 (3′-C), 50.8 (N′C(CH₃)₃), 44.6 (2-C), 41.1 (7-C), 40.0 (3′N—(CH₃)₂), 38.6 (4-C), 29.1 (N′C(C₃)₃), 28.9 (8-C), 27.7 (4′-C), 26.2 (6-CH₃), 21.2 (14-C), 20.6 (5′-CH₃), 19.4 (8-CH₃), 16.5 (12-CH₃), 16.0 (2-CH₃), 12.2 (10-CH₃), 10.6 (15-CH₃), 7.8 (4-CH₃).

### EXAMPLE 4

### 3-Decladinosyl-9-deoxo-9-dihydro-9a-N-(N′-benzylcarbamoyl)-9a-aza-9a-homoerythromycin A

To a solution of 3-decladinosyl-9-deoxo-9-dihydro-9a-aza-9a-homoerythromycin A (1.00 g) in toluene (20 ml) benzylisocyanate (0.25 g) was added. Reaction mixture was stirred for 1 hour at room temperature. Crude product (1.18 g) is obtained by filtration of precipitate from reaction mixture.

Crystallization from mixture acetone-petrol ether yielding 0.82 g of chromatographically homogenous title product with following physical-chemical constants:

TLC R_{f}=0.42 (CH₂Cl₂:MeOH=8:2)

IR (KBr) [ν/cm⁻¹]: 3404, 2975, 2936, 1731, 1625, 1529, 1454, 1384, 1349, 1319, 1274, 1172, 1111, 1091, 1049, 978, 957, 931, 900, 865, 837, 740, 700, 635.

MS m/z: (ES): MH⁺=710.6

¹H NMR (300 MHz, CDCl₃) [δ/ppm] 7.35 (4″-H, 6″-H), 7.30 (3″-H, 7″-H), 7.24 (5″-H), 5.03 (13-H), 4.55 (1″-Ha), 4.48 (9a-NCON′H), 4.30 (1′-H), 4.30 (1″-Hb), 3.89 (3-H), 3.68 (5′-H), 3.51 (11-H), 3.62 (5-H), 3.41 (9-Ha), 3.30 (2′-H), 2.62 (2-H), 2.30 (3′-H), 2.27 (3′-N(CH₃)₂), 1.89 (14-Ha), 1.69 (4′-Ha), 1.58 (14-Hb), 1.56 (4-H), 1.28 (4′-Hb), 1.30 (2-CH₃), 1.27 (5′-CH₃), 1.32 (10-CH₃), 1.28 (6-CH₃), 1.10 (12-CH₃), 1.04(8-CH₃), 0.93 (4-CH₃), 0.89 (15-CH₃).

¹³C NMR (75 MHz, CDCl₃) [δ/ppm] 176.9 (1-C), 159.6 (9a-NCONH), 139.4 (2″-C), 128.6 (4″-C, 6″-C), 127.3 (3″-C, 7″-C), 126.9 (5″-C), 105.7 (1′-C), 96.4 (5-C), 77.7 (13-C), 77.1 (12-C), 75.0 (11-C), 75.0 (3-C), 74.2 (6-C), 74.4 (9-C), 70.2 (5′-C), 69.2 (2′-C), 64.8 (3′-C), 44.7 (2-C), 44.6 (1″-C), 44.6 (7-C), 40.0 (3′N—(CH₃)₂), 38.4 (4-C), 27.5 (4′-C), 25.3 (6-CH₃), 21.3 (14-C), 20.7 (5′-CH₃), 19.7 (8-CH₃), 16.7 (12-CH₃), 15.9 (2-CH₃), 12.2 (10-CH₃), 10.4 (15-CH₃), 7.6 (4-CH₃).

### EXAMPLE 5

### 3-Decladinosyl-9-deoxo-9-dihydro-9a-N—[N-(3-trifluoromethylphenyl)carbamoyl]-9a-aza-9a-homoerythromycin A

To a solution of 3-decladinosyl-9-deoxo-9-dihydro-9a-aza-9a-homoerythromycin A (1.00 g) in toluene (20 ml) 3-trifluormetilphenylisocyanate (0.33 g) was added. Reaction mixture was stirred for 30 min at room temperature. The solvent was evaporated and 1.30 g of crude product is obtained.

Crystallization from mixture ethyl ether-petrol ether yielding 0.98 g of chromatographically homogenous title product with following physical-chemical constants:

TLC R_{f}=0.51 (CH₂Cl₂:MeOH=8:2)

IR (KBr) [ν/cm⁻¹]: 3451, 2975, 2935, 1727, 1704, 1659, 1548, 1494, 1449, 1384, 1336, 1258, 1167, 1125, 1072, 1049, 979, 957, 933, 901, 864, 835, 794, 758, 699, 659.

MS m/z: (ES): MH⁺=764.6

¹H NMR (300 MHz, CDCl₃) [δ/ppm] 7.70 (2″-H), 7.58 (6″-H), 7.35 (5″-H), 7.23 (4″-H), 4.82 (13-H), 4.62 (9a-NCON′H), 4.35 (1′-H), 3.87 (3-H), 3.62 (5′-H), 3.62 (5-H), 3.51 (11-H), 3.41 (9-Ha), 3.30 (2′-H), 2.66 (2-H), 2.50 (3′-H), 2.26 (3′-N(CH₃)₂), 1.88 (14-Ha), 1.71 (4′-Ha), 1.58 (14-Hb), 1.56 (4-H), 1.28 (6-CH₃), 1.28 (2-CH₃), 1.31 (4′-Hb), 1.27 (5′-CH₃), 1.24 (10-CH₃), 1.10 (12-CH₃), 1.04 (8-CH₃), 0.92 (4-CH₃), 0.88 (15-CH₃).

¹³C NMR (75 MHz, CDCl₃) [δ/ppm] 176.8 (1-C), 156.6 (9a-NCONH), 140.0 (1″-C), 131.3 (3″-C), 129.2 (5″-C), 122.3 (6″-C), 118.9 (4″-C), 115.8 (2″-C), 106.1 (1′-C), 96.5 (5-C), 78.8 (13-C), 77.1 (12-C), 75.7 (3-C), 74.8 (9-C), 74.7 (6-C), 74.6 (11-C), 70.6 (5′-C), 69.4 (2′-C), 65.2 (3′-C), 45.2 (2-C), 40.2 (3′N—(CH₃)₂), 38.8 (4-C), 27.9 (4′-C), 21.5 (14-C), 20.9 (5′-CH₃), 20.0 (8-CH₃), 16.8 (12-CH₃), 16.1 (2-CH₃), 13.8 (10-CH₃), 10.9 (15-CH₃), 7.7 (4-CH₃).

### EXAMPLE 6

### 3-Decladinosyl-9-deoxo-9-dihydro-9a-N—[N′-(2-trifluoromethylphenyl)carbamoyl]-9a-aza-9a-homoerythromycin A

To a solution of 3-decladinosyl-9-deoxo-9-dihydro-9a-aza-9a-homoerythromycin A (1.00 g) in toluene (20 ml) 2-trifluormetilphenylisocyanate (0.35 g) was added. Reaction mixture was stirred for 30 min at room temperature. The solvent was evaporated and 1.29 g of crude product is obtained.

Crystallization from mixture ethyl ether-petrol ether yielding 0.84 g of chromatographically homogenous title product with following physical-chemical constants:

TLC R_{f}=0.69 (CH₂Cl₂:MeOH=8:2)

IR (KBr) [ν/cm⁻¹]: 3458, 2975, 2940, 2879, 2786, 1727, 1658, 1591, 1536, 1457, 1384, 1322, 1282, 1244, 1171, 1112, 1035, 979, 956, 934, 901, 864, 834, 762, 702.

MS m/z: (FAB): MH⁺=764.3

¹H NMR (300 MHz, CDCl₃) [δ/ppm] 8.01 (6″-H), 7.55 (3″-H), 7.26 (4″-H), 7.16 (5″-H), 4.98 (13-H), 4.62 (9a-NCON′H), 4.38 (1′-H), 3.90 (3-H), 3.65 (5′-H), 3.64 (5-H), 3.61 (11-H), 3.31 (2′-H), 2.64 (2-H), 2.52 (3′-H), 2.27 (3′-N(CH₃)₂), 1.91 (14-Ha), 1.72 (4′-Ha), 1.57 (14-Hb), 1.56 (4-H), 1.35 (10-CH₃), 1.32 (2-CH₃), 1.28 (6-CH₃), 1.31 (4′-Hb), 1.24 (5′-CH₃), 1.15 (12-CH₃), 1.10 (8-CH₃), 0.96 (4-CH₃), 0.91 (15-CH₃).

¹³C NMR (75 MHz, CDCl₃) [δ/ppm] 177.3 (1-C), 157.0 (9a-NCONH), 136.4 (1″-C), 132.5 (3″-C), 125.7 (5″-C), 125.1 (4″-C), 123.4 (6″-C), 105.9 (1′-C), 82.9 (5-C), 77.9 (13-C), 74.4 (12-C), 75.2 (3-C), 74.4 (6-C), 73.8 (11-C), 70.4 (5′-C), 69.2 (2′-C), 64.9 (3′-C), 44.7 (2-C), 39.9 (3′N—(CH₃)₂), 38.6 (4-C), 27.6 (4′-C), 21.3 (14-C), 20.7 (5′-CH₃), 19.1 (8-CH₃), 16.6 (12-CH₃), 15.9 (2-CH₃), 12.5 (10-CH₃), 10.5 (15-CH₃), 7.7 (4-CH₃).

### EXAMPLE 7

### 3-Decladinosyl-9-deoxo-9-dihydro-9a-N—[N′-(3-trifluoromethylphenyl)thiocarbamoyl]-9a-aza-9a-homoerythromycin A

To a solution of 3-decladinosyl-9-deoxo-9-dihydro-9a-aza-9a-homoerythromycin A (1.00 g) in toluene (20 ml) 3-trifluormetilphenylisothiocyanate (0.35 g) was added. Reaction mixture was stirred for 15 min at room temperature. Crude product (1.18 g) is obtained by and filtration of precipitate from reaction mixture.

Crystallization from mixture acetone-petrol ether yielding 0.92 g of chromatographically homogenous title product with following physical-chemical constants:

TLC R_{f}=0.60 (CH₂Cl₂:MeOH=8:2)

IR (KBr) [ν/cm⁻¹]: 3449, 2975, 2937, 1708, 1600, 1547, 1494, 1452, 1384, 1328, 1255, 1164, 1116, 1073, 1019, 980, 956, 885, 862, 793, 735, 696.

MS m/z: (FAB): MH⁺=780

¹H NMR (300 MHz, CDCl₃) [δ/ppm] 7.68 (2″-H), 7.42 (6″-H), 7.26 (5″-H), 7.18 (4-H), 4.82 (13-H), 4.61 (9a-NCON′H), 4.40 (1′-H), 3.90 (3-H), 3.66 (5′-H), 3.57 (5-H), 3.49 (1-H), 3.31 (2′-H), 2.69 (2-H), 2.52 (3′-H), 2.27 (3′-N(CH₃)₂), 1.95 (14-Ha), 1.69 (4′-Ha), 1.62 (14-Hb), 1.53 (4-H), 1.34 (2-CH₃), 1.28 (6-CH₃), 1.34 (4′-Hb), 1.28 (5′-CH₃), 1.25 (10-CH₃), 1.21 (12-CH₃), 1.08 (8-CH₃), 0.98 (4-CH₃), 0.94 (15-CH₃).

¹³C NMR (75 MHz, CDCl₃) [δ/ppm] 183.2 (9a-NCONH), 177.6 (1-C), 140.4 (1″-C), 130.5 (3″-C), 130.2 (5″-C), 128.1 (6″-C), 125.2 (4″-C), 123.6 (2″-C), 105.2 (1′-C), 94.9 (5-C), 79.3 (3-C), 77.7 (13-C), 77.2 (12-C), 73.5 (9-C), 73.6 (6-C), 73.4 (11-C), 70.6 (5′-C), 69.5 (2′-C), 65.2 (3′-C), 44.5 (2-C), 41.1 (7-C), 40.2 (3′N—(CH₃)₂), 38.8 (4-C), 28.1 (8-C), 27.9 (4′-C), 25.9 (6-CH₃), 21.0 (14-C), 21.0 (5′-CH₃), 18.9 (8-CH₃), 16.6 (12-CH₃), 16.1 (2-CH₃), 12.8 (10-CH₃), 10.8 (15-CH₃), 7.6 (4-CH₃).

### EXAMPLE 8

### 3-Decladinosyl-9-deoxo-9-dihydro-9a-N—(N′-benzylthiocarbamoyl)-9a-aza-9a-homoerythromycin A

To a solution of 3-decladinosyl-9-deoxo-9-dihydro-9a-aza-9a-homoerythromycin A (1.00 g) in toluene (20 ml) benzylisothiocyanate (0.38 g) was added. Reaction mixture was stirred for 1 hour at room temperature. The solvent was evaporated and 1.35 g of crude product is obtained.

Crystallization from mixture ethyl ether-petrol ether yielding 1.06 g of chromatographically homogenous title product with following physical-chemical constants:

TLC R_{f}=0.40 (CH₂Cl₂:MeOH=8:2)

IR (KBr) [ν/cm⁻¹]: 3448, 2974, 2938, 1703, 1528, 1456, 1384, 1323, 1283, 1178, 1109, 1073, 1020, 978, 955, 934, 862, 825, 759, 699.

MS m/z: (ES): MH⁺=726.5

¹H NMR (300 MHz, CDCl₃) [δ/ppm] 7.34 (4″-H, 6″-H), 7.30 (3″-H, 7″-H), 7.27 (5″-H), 4.64 (9a-NCSN′H), 4.57 (13-H), 4.39 (1′-H), 3.89 (3-H), 3.67 (5′-H), 3.67 (5-H), 3.51 (11-H), 3.31 (2′-H), 2.64 (2-H), 2.55 (3′-H), 2.28 (3′-N(CH₃)₂), 1.93 (14-Ha), 1.73 (4′-Ha), 1.62 (14-Hb), 1.56 (4-H), 1.34 (2-CH₃), 1.27 (4′-Hb), 1.27 (5′-CH₃), 1.27 (10-CH₃), 1.25 (12-CH₃), 0.94 (8-CH₃), 0.87 (4-CH₃), 0.86 (15-CH₃).

¹³C NMR (75 MHz, CDCl₃) [δ/ppm] 189.2 (9a-NCSNH), 179.5 (1-C), 137.5 (2″-C), 128.6 (4″-C, 6″-C), 128.2 (3″-C, 7″-C), 127.6 (5″-C), 105.9 (1′-C), 93.9 (5-C), 78.7 (13-C), 75.1 (12-C), 75.2 (6-C), 74.6 (3-C), 73.9 (11-C), 70.1 (5′-C), 69.5 (2′-C), 65.2 (3′-C), 50.8 (1″-C), 44.1 (2-C), 40.2 (3′N—(CH₃)₂), 44.4 (4-C), 27.9 (4′-C), 21.1 (14-C), 20.9 (5′-CH₃), 19.0 (8-Me), 16.7 (12-CH₃), 16.5 (2-CH₃), 12.3 (10-CH₃), 11.3 (15-CH₃), 8.3 (4-CH₃).

### EXAMPLE 9

### 3-Decladinosyl-9-deoxo-9-dihydro-9a-N—[N′-(2,4-dichlorophenyl)carbamoyl]-9a-aza-9a-homoerythromycin A

To a solution of 3-decladinosyl-9-deoxo-9-dihydro-9a-aza-9a-homoerythromycin A (3.00 g) in toluene (50 ml) 2,4-dichlorophenylisocyanate (1.07 g) was added. Reaction mixture was stirred for 1 hour at room temperature. The solvent was evaporated and 3.80 g of crude product is obtained.

Crystallization from mixture ethyl ether-petrol ether yielding 4.26 g of chromatographically homogenous title product with following physical-chemical constants:

TLC R_{f}=0.63 (CH₂Cl₂:MeOH=8:2)

IR (KBr) [ν/cm⁻¹]: 3428, 2973, 2934, 2878, 1718, 1659, 1580, 1517, 1459, 1382, 1345, 1297, 1229, 1176, 1110, 1086, 1048, 978, 955, 932, 901, 866, 825, 759, 731, 696, 631.

MS m/z: (ES): MH⁺=764.5

¹H NMR (300 MHz, CDCl₃) [δ/ppm] 8.17 (6″-H), 7.33 (3″-H), 7.17 (5″-H), 4.71 (13-H), 4.57 (9a-NC0N′H), 4.40 (1′-H), 4.10 (5-H), 3.89 (3-H), 3.89 (5′-H), 3.32 (2′-H), 2.62 (2-H), 2.60 (3′-H), 2.30 (3′-N(CH₃)₂), 1.93 (14-Ha), 1.76 (4′-Ha), 1.57 (14-Hb), 1.56 (4-H), 1.53 (12-CH₃), 1.36 (10-CH₃), 1.31 (2-CH₃), 1.29 (4′-Hb), 1.29 (8-CH₃), 1.27 (5′-CH₃), 0.94 (4-CH₃), 0.90 (15-CH₃).

¹³C NMR (75 MHz, CDCl₃) [δ/ppm] 177.2 (1-C), 156.4 (9a-NCONH), 134.7 (1″-C), 128.4 (3″-C), 127.6 (2″-C), 125.3 (5″-C), 123.2 (4″-C), 105.9 (1′-C), 78.2 (13-C), 75.9 (3-C), 70.5 (5′-C), 69.5 (2′-C), 65.2 (3′-C), 44.9 (2-C), 40.2 (3′N—(CH₃)₂), 38.8 (4-C), 28.1 (4′-C), 28.0 (8-C), 21.5 (14-C), 20.9 (5′-CH₃), 19.4 (6-CH₃), 16.8 (12-CH₃), 16.2 (2-CH₃), 12.9 (10-CH₃), 10.9 (15-CH₃), 8.0 (4-CH₃).

### EXAMPLE 10

### 3-Decladinosyl-9-deoxo-9-dihydro-9a-N—[N′-allylthiocarbamoyl)-9a-aza-9a-homoerythromycin A

To a solution of 3-decladinosyl-9-deoxo-9-dihydro-9a-aza-9a-homoerythromycin A (1.00 g) in toluene (20 ml) allylisothiocyanate (0.19 g) was added. Reaction mixture was stirred for 1 hour at room temperature. The solvent was evaporated and 1.20 g of crude product is obtained.

Crystallization from mixture ethyl ether-petrol ether yielding 0.72 g of chromatographically homogenous title product with following physical-chemical constants:

TLC R_{f}=0.68 (CH₂Cl₂:MeOH=8:2)

IR (KBr) [ν/cm⁻¹]: 3451, 2974, 2938, 2878, 1704, 1644, 1520, 1456, 1384, 1321, 1286, 1178, 1110, 1074, 1048, 1019, 978, 955, 934, 862, 833, 762, 632.

MS m/z: (ES): MH⁺=676.4

¹H NMR (300 MHz, CDCl₃) [δ/ppm] 5.94 (2″-H), 5.29 (3″-Ha), 5.20 (3″-Hb), 4.57 (13-H), 4.41 (1′-H), 4.37 (9a-NCSN′H), 4.34 (1″-Ha), 4.21 (1″-Hb), 3.90 (3-H), 3.69 (5′-H), 3.78 (5-H), 3.32 (2′-H), 2.64 (2-H), 2.54 (3′-H), 2.28 (3′-N(CH₃)₂), 1.92 (14-Ha), 1.70 (4′-Ha), 1.60 (14-Hb), 1.52 (4-H), 1.34 (4′-Hb), 1.35 (2-CH₃), 1.28 (5′-CH₃), 1.28 (6-CH₃), 1.26 (10-CH₃), 1.24 (12-CH₃), 1.12 (8-CH₃), 0.95 (4-CH₃), 0.94 (15-CH₃).

¹³C NMR (75 MHz, CDCl₃) [δ/ppm] 189.3 (1-C), 179.7 (9a-NCSNH), 133.6 (2′-C), 117.8 (3″-C), 106.0 (1′-C), 95.0 (5-C), 78.6 (13-C), 75.1 (12-C), 75.1 (6-C), 74.8 (3-C), 73.8 (111-C), 70.4 (5′-C), 69.3 (2′-C), 65.4 (3′-C), 48.8 (1″-C), 44.2 (2-C), 39.9 (3′N—(CH₃)₂), 38.9 (4-C), 27.6 (4′-C), 20.7 (14-C), 20.6 (5′-CH₃), 18.7 (8-CH₃), 16.4 (12-CH₃), 16.2 (2-CH₃), 12.1 (10-CH₃), 10.9 (15-CH₃), 7.9 (4-CH₃).

### EXAMPLE 11

### 2′-O-Acetyl-3-decladinosyl-9-deoxo-9-dihydro-9a-N-(N′-isopropylcarbamoyl)-9a-aza-9a-homoerythromycin A

To a solution of 3-decladinosyl-9-deoxo-9-dihydro-9a-N-(N-isopropylcarbamoyl)-9a-aza-9a-homoerythromycin A (1.00 g) in CH₂Cl₂ (20 ml), NaHCO₃ (1.01 g) and acetic acid anhydride (173 μl) were added and it was then stirred for 24 hours at room temperature. Onto the reaction mixture a saturated NaHCO₃ solution was added, the layers were separated and the aqueous one was extracted two more times with CH₂Cl₂. The combined organic extracts were rinsed with saturated NaHCO₃ solution and water and evaporated yielding the title product (1.10 g) with following physical-chemical constants:

TLC R_{f}=0.71 (CH₂Cl₂:MeOH=8:2).

IR (KBr) [νcm⁻¹]: 3423, 2974, 2938, 2878, 1735, 1624, 1560, 1522, 1459, 1376, 1252, 1169, 1109, 1058, 987, 956, 901, 834, 771, 670.

MS m/z: (ES): MH⁺=704.2

### EXAMPLE 12

### 2′-O-Acetyl-3-O-mesyl-3-decladinosyl-9-deoxo-9-dihydro-9a-N-(N′-isopropylcarbamoyl)-9a-aza-9a-homoerythromycin A

To a solution of 2′-O-Acetyl-3-decladinosyl-9-deoxo-9-dihydro-9a-N—(N′-isopropylcarbamoyl)-9a-aza-9a-homoerythromycin A (1.00 g) in pyridine (5 ml) methylsulphonyl anhydride (0.30 g) was added and the reaction mixture was stirred at room temperature for 3 hours. The solvent was evaporated and the residue was dissolved in CH₂Cl₂ (20 ml). Saturated aqueous solution of NaHCO₃ (20 ml) was added, the layers were separated and the water layer was extracted two more times with CH₂Cl₂ Combined organic extracts were rinsed with NaHCO₃ and brine, dried over K₂CO₃ and evaporated yielding 0.87 g of crude product with following physical-chemical constants

TLC R_{f}=0.43 (CHCl₃:MeOH:NH₄OH=6:1:0.1).

IR (KBr) [νcm⁻¹]: 3422, 3065, 2972, 2876, 1735, 1637, 1618, 1535, 1487, 1459, 1377, 1330, 1240, 1206, 1193, 1059, 1004, 785, 773, 755, 682, 609.

MS m/z: (ES): MH⁺=783.2

### EXAMPLE 13

### 3-Decladinosyl-3,6-hemiketal-9-deoxo-9-dihydro-9a-N-(A,-isopropylcarbamoyl)-9a-aza-9a-homoerythromycin A

To a solution of 2′-O-Acetyl-3-decladinosyl-9-deoxo-9-dihydro-9a-N-(N′-isopropylcarbamoyl)-9a-aza-9a-homoerythromycin A (1.50 g) from Example 11. in CH₂Cl₂ (30 ml) dimethylsulfoxide (2.00 ml) and N,N-dimethyl-aminopropyl-ethyl-carbodiimide (2.30 ml) were added. The reaction mixture was cooled to 150° C., a then, keeping the temperature constant, solution of pyridinium trifluoracetate (2.30 g) in CH₂Cl₂ (10 ml) was added dropwise during 30 minutes. The reaction mixture was stirred at 150° C. to room temperature for additional 2 hours. To the reaction mixture saturated aqueous solution of NaCl (40 ml) was added and the pH value was adjusted to pH 9.5. The layers were separated and the water layer was extracted two more times with CH₂Cl₂. Combined organic extracts were rinsed with brine, NaHCO₃ and water, dried over K₂CO₃ and evaporated yielding 1.36 g of the crude product which is dissolved in MeOH (50 ml) and stirred for 24 hours at room temperature. The solvent was evaporated and the residue purified by low pressure chromatography on a silica gel column using the system CH₂Cl₂:MeOH:NH₄OH=90:3:0.3. The combining and evaporating of chromatographically homogenous fractions gave the title product (0.313 g) with following physical-chemical constants:

TLC R_{f}=0.51 (CH₂Cl₂:MeOH:NH₄OH=90:9:0.5).

IR (KBr) [νcm⁻¹]: 3373, 2968, 2936, 2877, 1728, 1716, 1615, 1526, 1520, 1456, 1384, 1261, 1181, 1099, 1031, 961, 864, 799, 737.

MS m/z: (ES): MH⁺=660.6

¹H NMR (300 MHz, CDCl₃) [δ/ppm] 4.91 (13-H), 4.21 (1′-H), 4.20 (9a-NCON′H), 3.96 (1″-H), 3.63 (11-H), 3.53 (5′-H), 3.78 (5-H), 3.26 (2′-H), 2.64 (2-H), 2.59 (10-H), 2.54 (3′-H), 2.32 (3′-N(CH₃)₂), 2.08 (4-H), 2.06 (8-H), 1.82 (14-Ha), 1.73 (4′-Ha), 1.62 (14-Hb), 1.48 (7-Ha), 1.39 (6-CH₃), 1.36 (7-Hb), 1.28 (10-CH₃), 1.27 (4′-Hb), 1.24 (5′-CH₃), 1.24 (4-CH₃), 1.15 (12-CH₃), 1.13 (1″-(CH₃)₂), 0.97 (8-CH₃), 0.96 (2-CH₃), 0.87 (15-CH₃).

¹³C NMR (75 MHz, CDCl₃) [δ/ppm] 176.4 (1-C), 157.4 (9a-NCONH), 103.7 (1′-C), 103.1 (3-C), 94.2 (5-C), 83.8 (6-C), 83.3 (11-C), 79.0 (13-C), 75.4 (12-C), 69.7 (5′-C), 69.6 (2′-C), 65.4 (3′-C), 49.2 (2-C), 48.3 (4-C), 46.5 (10-C), 42.6 (1″-C), 41.4 (7-C), 40.4 (3′-N(CH₃)₂), 29.0 (4′-C), 28.8 (8-C), 26.3 (6-CH₃), 23.6 (1″-(CH₃)₂), 21.6 (14-C), 21.2 (5′-CH₃), 21.2 (8-CH₃), 17.7 (12-CH₃), 14.3 (10-CH₃), 13.9 (2-CH₃), 12.9 (4-CH₃), 10.8 (15-CH₃).

### EXAMPLE 14

### 3-Decladinosyl-3,6-cyclic ether-9-deoxo-9-dihydro-9a-N—(N′-isopropylcarbamoyl)-9a-aza-9a-homoerythromycin A

To a solution of 2′-O-Acetyl-3-O-mesyl-3-decladinosyl-9-deoxo-9-dihydro-9a-N—(N′-isopropylcarbamoyl)-9a-aza-9a-homoerythromycin A from Example 12. (0.80 g) in DMF/THF (3.5 ml/1 ml) suspension (60%) of NaH in mineral oil (163 mg) was added and the reaction mixture was stirred at 0° C. for 5 hours, and additional 20 h at the room temperature. The reaction mixture was poured into saturated aqueos solution of NaHCO₃ (20 ml), EtOAc (20 ml) was added and the layers were separated. The water layer was extracted two more times with EtOAc. Combined organic extracts were rinsed with NaHCO₃ and brine, dried over K₂CO₃ and evaporated yielding 0.53 g of product. The obtained product was dissolved in MeOH (20 ml) was stirred for 24 hours at room temperature. The solvent was evaporated and the crude product was purified by chromatography on a silica gel column using the system CH₂Cl₂:MeOH:NH₄OH=90:3:0.3. The combining and evaporating of chromatographically homogenous fractions gave the title product (0.17 g) with following physical-chemical constants:

TLC R_{f}0.32 (CH₂Cl₂:MeOH:NH₄OH=90:9:0.5).

IR (KBr) [νcm⁻¹]: 3428, 2972, 2938, 2876, 1735, 1619, 1528, 1459, 1383, 1330, 1270, 1179, 1144, 1073, 1051, 1030, 961, 893, 835, 799, 759, 635.

MS m/z: (ES): MH⁺=644.2

¹H NMR (300 MHz, CDCl₃) [(δ/ppm] 4.90 (13-H), 4.24 (1′-H), 4.20 (9a-NCON′H), 3.95 (11-H), 3.83 (11-H), 3.64 (3-H), 3.55 (5′-H), 3.57 (5-H), 3.33 (2′-H), 3.25 (9-Ha), 2.92 (9-Hb), 2.76 (3′-H), 2.57 (2-H), 2.48 (3′-N(CH₃)₂), 2.06 (8-H), 2.04 (4-H), 1.92 (4′-Ha), 1.83 (14-Ha), 1.64 (14-Hb), 1.64 (7-Ha), 1.33 (4′-Hb), 1.29 (7-Hb), 1.28 (10-CH₃), 1.26 (5′-CH₃), 1.24 (4-CH₃), 1.23 (6-CH₃), 1.19 (2-CH₃), 1.14 (12-CH₃), 1.13 (1″-(CH₃)₂), 0.96 (8-CH₃), 0.88 (15-CH₃).

¹³C NMR (75 MHz, CDCl₃) [δ/ppm] 176.6 (1-C), 157.8 (9a-NCONH), 103.9 (1′-C), 93.4 (5-C), 84.0 (6-C), 83.6 (3-C), 78.7 (13-C), 75.5 (12-C), 69.5 (2′-C), 69.4 (5′-C), 65.4 (3′-C), 46.5 (2-C), 45.7 (4-C), 42.6 (1″-C), 40.5 (3′-N(CH₃)₂), 40.3 (7-C), 30.1 (4′-C), 28.9 (8-C), 23.6 (1″-(CH₃)₂), 23.1 (6-CH₃), 21.7 (14-C), 21.3 (8-CH₃), 21.1 (5′-CH₃), 18.0 (12-CH₃), 18.0 (4-CH₃), 14.3 (2-CH₃), 12.8 (10-CH₃), 11.0 (15-CH₃).

### EXAMPLE 15

### 3-Decladinosyl-3-O-(4-nitrophenyl)acyl-9-deoxo-9-dihydro-9a-N—(N′-isopropylcarbamoyl)-9a-aza-9a-homoerythromycin A

To a solution of 4-nitrophenylacetic acid (0.85 g) in dry CH₂Cl₂ (25 ml) TEA (0.65 ml) was added and the reaction mixture was cooled to 2-5° C. Pyvaloyl chloride (0.57 ml) was added and the reaction mixture was stirred at the same temperature for 30 minutes. To a reaction mixture pyridine (1.27 ml) and the solution of 2′-O-Acetyl-3-decladinosyl-9-deoxo-9-dihydro-9a-N—(N′-isopropylcarbamoyl)-9a-aza-9a-homoerythromycin A from Example 11 (1.00 g) in dry CH₂Cl₂ (5 ml) were added and the reaction mixture was stirred at room temperature for 20 hours. Saturated aqueous solution of NaHCO₃ (30 ml) was added and the layers were separated. The water layer was extracted two more times with CH₂Cl₂. Combined organic extracts were rinsed with brine, dried over K₂CO₃ and evaporated yielding 1.429 g of oily product. The obtained product was dissolved in MeOH (50 ml) was stirred for 24 hours at room temperature. The solvent was evaporated and the crude product was purified by chromatography on a silica gel column using the system CH₂Cl₂:MeOH:NH₄OH=90:3:0.3. The combining and evaporating of chromatographically homogenous fractions gave the title product (0.45 g) with following physical-chemical constants:

TLC R_{f}0.40 (CH₂Cl₂:MeOH:NH₄OH=90:9:0.5).

IR (KBr) [νcm⁻¹]: 3449, 2975, 2939, 2877, 2791, 1741, 1626, 1604, 1523, 1459, 1382, 1347, 1255, 1167, 1111, 1075, 1051, 1032, 984, 959, 856, 767, 728, 687.

MS m/z: (ES): MH⁺=825

¹H NMR (300 MHz, CDCl₃) [δ/ppm] 8.21 (4′″-H, 6′″-H), 7.54 (3′″-H, 7′″-H), 5.43 (3-H), 5.06 (13-H), 4.42 (9a-NCON′H), 4.23 (1′-H), 3.92 (1′″-H), 3.60 (5-H), 3.49 (5′-H), 3.26 (2′-H), 2.68 (2-H), 2.45 (3′-H), 2.29 (3′-N(CH₃)₂), 2.20 (8-H), 1.95 (14-Ha), 1.90 (4-H), 1.66 (4′-Ha), 1.50 (14-Hb), 1.32 (6-CH₃), 1.26 (4′-Hb), 1.29 (10-CH₃), 1.22 (5′-CH₃), 1.17 (12-CH₃), 1.05 (4-CH₃), 0.95 (2-CH₃), 1.15 (1″-(CH₃)₂), 1.08 (8-CH₃), 0.87 (15-CH₃).

¹³C NMR (75 MHz, CDCl₃) [δ/ppm] 174.7 (1-C), 169.9 (1″-CO), 158.7 (9a-NCONH), 147.1 (5′″-C), 141.5 (2′″-C), 130.5 (3′″-C, 7′″-C), 123.6 (2′″-C, 6′″-C), 103.4 (1′-C), 88.3 (5-C), 78.1 (13-C), 74.5 (3-C), 74.2 (6-C), 70.4 (2′-C), 69.8 (5′-C), 65.4 (3′-C), 44.6 (2-C), 41.3 (1′″-C), 38.4 (4-C), 48.7 (1″-C), 41.3 (7-C), 40.2 (3′-N(CH₃)₂), 28.7 (4′-C), 27.8 (8-C), 27.4 (6-CH₃), 23.2 (1″-(CH₃)₂), 21.7 (14-C), 20.7 (8-CH₃), 20.8 (5′-CH₃), 16.9 (12-CH₃), 15.2 (2-CH₃), 12.5 (10-CH₃), 11.0 (15-CH₃), 8.9 (4-CH₃).

### EXAMPLE 16

### 2′-O-Acetyl-3-decladinosyl-9-deoxo-9-dihydro-9a-N—[N′-(2,4-dichlorophenyl)carbamoyl]-9a-aza-9a-homoerythromycin A

To a solution of 3-decladinosyl-9-deoxo-9-dihydro-9a-N—[N′-(2,4-dichlorophenyl)carbamoyl]-9a-aza-9a-homoerythromycin A (6.60 g) in CH₂Cl₂ (250 ml), NaHCO₃ (3.25 g) and acetic acid anhydride (895 μl) were added and it was then stirred for 24 hours at room temperature. In to the reaction mixture a saturated NaHCO₃ solution was added, the layers were separated and the aqueous one was extracted two more times with CH₂Cl₂. The combined organic extracts were rinsed with saturated NaHCO₃ solution and water and evaporated yielding the title product (6.30 g) with following physical-chemical constants:

TLC R_{f}=0.33 (CH₂Cl₂:MeOH:NH₄OH=90:9:1.5).

IR (KBr) [νcm⁻¹]: 3545, 3448, 3393, 2972, 2940, 2882, 2831, 2787, 1727, 1638, 1586, 1522, 1490, 1460, 1382, 1335, 1309, 1298, 1247, 1201, 1166, 1100, 1057, 1036, 1006, 985, 947, 893, 864, 817, 756, 702, 669, 620.

MS m/z: (ES): MH⁺=806.16

### EXAMPLE 17

### 2′-O-Acetyl-3-O-mesyl-3-decladinosyl-9-deoxo-9-dihydro-9a-N—[N′-(2,4-dichlorophenyl)carbamoyl]-9a-aza-9a-homoerythromycin A

To a solution of 2′-O-Acetyl-3-decladinosyl-9-deoxo-9-dihydro-9a-N—[N′-(2,4-dichlorophenyl)carbamoyl]-9a-aza-9a-homoerythromycin A (1.50 g) in pyridine (60 ml) methylsulphonyl anhydride (1.16 g) was added and the reaction mixture was stirred at room temperature for 3 hours. The solvent was evaporated and the residue was dissolved in CH₂Cl₂ (50 ml). Saturated aqueous solution of NaHCO₃ (50 ml) was added, the layers were separated and the water layer was extracted two more times with CH₂Cl₂ Combined organic extracts were rinsed with NaHCO₃ and brine, dried over K₂CO₃ and evaporated yielding 1.62 g of crude product with following physical-chemical constants

TLC R_{f}=0.69 (etihylacetate:hexane:diethylamine=10:10:2).

IR (KBr) [νcm⁻¹]: 3448, 3058, 2935, 1735, 1655, 1637, 1603, 1560, 1528, 1486, 1376, 1332, 1240, 1208, 1193, 1097, 1059, 1001, 916, 822, 785, 773, 753, 681, 609.

MS m/z: (ES): MH⁺=884.05

### EXAMPLE 18

### 3-Decladinosyl-3,6-hemiketal-9-deoxo-9-dihydro-9a-N-[N′(2,4-dichlorophenyl)carbamoyl]-9a-aza-9a-homoerythromycin A

To a solution of 2′-O-Acetyl-3-decladinosyl-9-deoxo-9-dihydro-9a-N—[N′-(2,4-dichlorophenyl)carbamoyl]-9a-aza-9a-homoerythromycin A (2.00 g) from Example 16. in CH₂Cl₂ (30 ml) dimethylsulfoxide (2.66 ml) and N,N-dimethyl-aminopropyl-ethyl-carbodiimide (2.85 ml) were added. The reaction mixture was cooled to 150° C., a then, keeping the temperature constant, solution of pyridinium trifluoracetate (2.15 g) in CH₂Cl₂ (10 ml) was added drop wise during 30 minutes. The reaction mixture was stirred at 15° C. to room temperature for additional 2 hours. To the reaction mixture saturated aqueous solution of NaCl (40 ml) was added and the pH value was adjusted to pH 9.5. The layers were separated and the water layer was extracted two more times with CH₂Cl₂ Combined organic extracts were rinsed with brine, NaHCO₃ and water, dried over K₂CO₃ and evaporated yielding 1.10 g of the crude product which is dissolved in MeOH (50 ml) and stirred for 24 hours at room temperature. The solvent was evaporated and the residue purified by low pressure chromatography on a silica gel column using the system CH₂Cl₂:MeOH:NH₄OH=90:3:0.3. The combining and evaporating of chromatographically homogenous fractions gave the title product (0.516 g) with following physical-chemical constants:

TLC R_{f}=0.60 (CH₂Cl₂:MeOH:NH₄OH=90:9:0.5).

IR (KBr) [νcm⁻¹]: 3448, 2974, 2938, 2877, 2782, 1720, 1667, 1580, 1512, 1460, 1384, 1324, 1299, 1231, 1195, 1115, 1099, 1072, 1049, 962, 862, 823.

MS m/z: (ES): MH⁺=762.12

¹H NMR (300 MHz, CDCl₃) [δ/ppm] 8.14 (6″-H), 7.32 (3″-H), 7.19 (5″-H), 4.91 (13-H), 4.36 (9a-NC0N′H), 4.21 (1′-H), 3.80 (5-H), 3.63 (11-H), 3.51 (5′-H), 3.23 (2′-H), 2.59 (10-H), 2.56 (2-H), 2.49 (3′-H), 2.27 (3′-N(CH₃)₂), 2.20 (8-H), 2.09 (4-H), 1.83 (14-Ha), 1.68 (4′-Ha), 1.62 (14-Hb), 1.55 (7-Ha), 1.47 (7-Hb), 1.42 (6-CH₃), 1.30 (2-CH₃), 1.26 (4′-Hb), 1.26 (5′-CH₃), 1.24 (4-CH₃), 1.21 (12-CH₃), 1.19 (10-CH₃), 1.07 (8-CH₃), 0.88 (15-CH₃).

¹³C NMR (75 MHz, CDCl₃) [δ/ppm] 177.4 (1-C), 154.8 (9a-NCONH), 134.8 (1″-C), 128.3 (3″-C), 127.3 (5″-C), 127.1 (2″-C), 122.7 (4″-C), 121.9 (6″-C), 106.2 (1′-C), 103.3 (3-C), 94.2 (5-C), 83.6 (6-C), 83.3 (11-C), 79.0 (13-C), 75.6 (12-C), 69.7 (2′-C), 69.6 (5′-C), 65.5 (3′-C), 49.2 (2-C), 48.4 (4-C), 46.3 (10-C), 41.4 (7-C), 40.3 (3′N—(CH₃)₂), 28.6 (4′-C), 28.4 (8-C), 21.6 (14-C), 23.3 (5′-CH₃), 26.4 (6-CH₃), 21.5 (8-CH₃), 17.7 (12-CH₃), 16.8 (12-CH₃), 14.2 (10-CH₃), 13.8 (2-CH₃), 12.9 (4-CH₃), 10.9 (15-CH₃).

### EXAMPLE 19

### 3-Decladinosyl-3,6-cyclic ether-9-deoxo-9-dihydro-9a-N-[N′-(2,4-dichlorophenyl)carbamoyl]-9a-aza-9a-homoerythromycin A

To a solution of 2′-O-Acetyl-3-O-mesyl-3-decladinosyl-9-deoxo-9-dihydro-9a-N—[N′-(2,4-dichlorophenyl)carbamoyl]-9a-aza-9a-homoerythromycin A from Example 12. (1.50 g) in DMF/THF (57 ml/19 ml) suspension (60%) of NaH in mineral oil (270 mg) was added and the reaction mixture was stirred at 0° C. for 5 hours, and additional 20 h at the room temperature. The reaction mixture was poured into saturated aqueous solution of NaHCO₃ (30 ml), EtOAc (30 ml) was added and the layers were separated. The water layer was extracted two more times with EtOAc. Combined organic extracts were rinsed with NaHCO₃ and brine, dried over K₂CO₃ and evaporated yielding 1.25 g of product. The obtained product was dissolved in MeOH (20 ml) was stirred for 24 hours at room temperature. The solvent was evaporated and the crude product was purified by chromatography on a silica gel column using the system CH₂Cl₂:MeOH:NH₄OH=90:3:0.3. The combining and evaporating of chromatographically homogenous fractions gave the title product (0.27 g) with following physical-chemical constants:

TLC R_{f}=0.42 (CH₂Cl₂:MeOH:NE₄OH=90:9:0.5).

IR (KBr) [νcm⁻¹]: 3448, 2971, 2936, 2875, 2782, 1736, 1670, 1579, 1510, 1460, 1383, 1327, 1299, 1260, 1177, 1142, 1115, 1099, 1072, 1047, 961, 890, 861, 829, 763, 670, 630.

MS m/z: (ES): MH⁺=746.02

¹H NMR (300 MHz, CDCl₃) [δ/ppm] 8.17 (6″-H), 7.32 (3″-H), 7.18 (5″-H), 4.91 (13-H), 4.23 (9a-NC0N′H), 4.20 (1′-H), 3.88 (11-H), 3.56 (5-H), 3.51 (5′-H), 3.18 (2′-H), 2.59 (2-H), 2.52 (3′-H), 2.29 (3′-N(CH₃)₂), 2.18 (8-H), 2.06 (4-H), 1.84 (14-Ha), 1.71 (14-Hb), 1.69 (4′-Ha), 1.63 (7-Ha), 1.44 (4′-Hb), 1.28 (10-CH₃), 1.27 (7-Hb), 1.25 (6-CH₃), 1.23 (4-CH₃), 1.21 (5′-CH₃), 1.20 (2-CH₃), 1.13 (12-CH₃), 1.06 (8-CH₃), 0.89 (15-CH₃).

¹³C NMR (75 MHz, CDCl₃) [δ/ppm] 176.1 (1-C), 154.2 (9a-NCONH), 134.4 (1″-C), 127.6 (3″-C), 127.1 (5″-C), 126.4 (2″-C), 122.1 (4″-C), 121.2 (6″-C), 104.3 (1′-C), 93.4 (5-C), 83.3 (11-C), 83.2 (6-C), 82.8 (3-C), 79.1 (13-C), 74.9 (12-C), 69.1 (5′-C), 69.0 (2′-C), 64.9 (3′-C), 45.7 (2-C), 45.2 (4-C), 40.1 (7-C), 39.7 (3′N—(CH₃)₂), 30.0 (4′-C), 29.2 (8-C), 21.2 (14-C), 21.0 (5′-CH₃), 22.7 (6-CH₃), 20.9 (8-CH₃), 12.6 (4-CH₃), 17.0 (12-CH₃), 12.8 (10-CH₃), 13.7 (2-CH₃), 10.4 (15-CH₃).

### EXAMPLE 20

### 3-Decladinosyl-3-O-(4-nitrophenyl)acyl-9-deoxo-9-dihydro-9a-N-[N′(2,4-dichlorophenyl)carbamoyl]-9a-aza-9a-homoerythromycin A

To a solution of 4-nitrophenylacetic acid (0.74 g) in dry CH₂Cl₂ (25 ml) TEA (0.57 ml) was added and the reaction mixture was cooled to 2-5° C. Pyvaloyl chloride (0.50 ml) was added and the reaction mixture was stirred at the same temperature for 30 minutes. To a reaction mixture pyridine (1.1 ml) and the solution of 2′-O-Acetyl-3-decladinosyl-9-deoxo-9-dihydro-9a-N—[N′-(2,4-dichlorophenyl)carbamoyl]-9a-aza-9a-homoerythromycin A from Example 16 (1.00 g) in dry CH₂Cl₂ (5 ml) were added and the reaction mixture was stirred at room temperature for 20 hours. Saturated aqueous solution of NaHCO₃ (30 ml) was added and the layers were separated. The water layer was extracted two more times with CH₂Cl₂. Combined organic extracts were rinsed with brine, dried over K₂CO₃ and evaporated yielding 1.14 g of oily product. The obtained product was dissolved in MeOH (50 ml) was stirred for 24 hours at room temperature. The solvent was evaporated and the crude product was purified by chromatography on a silica gel column using the system CH₂Cl₂:MeOH:NH₄OH=90:3:0.3. The combining and evaporating of chromatographically homogenous fractions gave the title product (0.52 g) with following physical-chemical constants:

TLC R_{f}=0.55 (CH₂Cl₂:MeOH:NH₄OH=90:9:0.5).

IR (KBr) [νcm⁻¹]: 3448, 2975, 2938, 2879, 2789, 1741, 1665, 1607, 1578, 1522, 1459, 1382, 1347, 1298, 1256, 1229, 1165, 1110, 1074, 1050, 983, 958, 857, 821, 731, 685, 669.

MS m/z: (ES): MH⁺=927.2

¹H NMR (300 MHz, CDCl₃) [δ/ppm] 8.18 (4′″-H, 6′″-H), 8.13 (6″-H), 7.51 (3′″-H, 7′″-H), 7.32 (3″-H), 7.18 (5″-H), 5.36 (3-H), 5.03 (13-H), 4.42 (9a-NCON′H), 4.23 (1′-H), 3.80 (1′″-H), 3.69 (5-H), 3.49 (5′-H), 3.25 (2′-H), 2.68 (2-H), 2.45 (3′-H), 2.32 (3′-N(CH₃)₂), 2.29 (8-H), 1.92 (14-Ha), 1.92 (4-H), 1.66 (4′-Ha), 1.58 (7-Ha), 1.51 (14-Hb), 1.38 (10-CH₃), 1.32 (6-CH₃), 1.26 (7-Hb), 1.18 (4′-Hb), 1.24 (12-CH₃), 1.16 (5′-CH₃), 1.07 (8-CH₃), 0.95 (2-CH₃), 0.94 (4-CH₃), 0.85 (15-CH₃).

¹³C NMR (75 MHz, CDCl₃) [δ/ppm] 174.9 (1-C), 169.9 (1″-CO), 154.9 (9a-NCONH), 147.2 (5′″-C), 141.3 (2′″-C), 134.8 (1″-C), 130.6 (3′″-C, 7′″-C), 128.5 (3″-C), 127.4 (2″-C, 5″-C), 123.7 (2′″-C, 6′″-C), 122.6 (4″-C), 122.2 (6″-C), 103.9 (1′-C), 88.3 (5-C), 77.6 (13-C), 74.3 (3-C), 74.2 (6-C), 70.5 (2′-C), 69.8 (5′-C), 65.5 (3′-C), 47.3 (4-C), 44.6 (2-C), 41.2 (1′″-C), 41.2 (7-C), 40.5 (3′-N(CH₃)₂), 29.1 (4′-C), 27.5 (8-C), 27.4 (6-CH₃), 22.0 (14-C), 21.1 (5′-CH₃), 21.0 (8-CH₃), 12.3 (12-CH₃), 13.1 (10-CH₃), 11.2 (15-CH₃), 3.2 (4-CH₃).

### EXAMPLE 21

### 3-Decladinosyl-6-O-Me-9-deoxo-9-dihydro-9a-N—(N′-isopropylcarbamoyl)-9a-aza-9a-homoerythromycin A

To a solution of 3-decladinosyl-6-O-Me-9-deoxo-9-dihydro-9a-aza-9a-homoerythromycin A (0.20 g) in acetonitrile (20 ml) isopropylisocyanate (0.07 ml) was added. Reaction mixture was stirred for 2 hours at room temperature. The solvent was evaporated and 0.25 g of crude product is obtained. Crude product was purified by chromatography on a silica gel column using the system (CH₂Cl₂:MeOH:NH₄OH=90:5:0.5). The combining and evaporating of chromatographically homogenous fractions gave the title product (0.128 g) with following physical-chemical constants:

TLC R_{f}=0.54 (CHCl₃:MeOH:NH₄OH=6:1:0.1).

IR (KBr) [νcm⁻¹]: 3424, 2975, 2937, 2877, 1732, 1687, 1627, 1562, 1525, 1460, 1379, 1270, 1166, 1112, 1080, 1053, 984, 958, 938, 896, 828, 766.

MS m/z: (ES): MH⁺=676.8

¹H NMR (300 MHz, CDCl₃) [δ/ppm] 5.16 (13-H), 4.44 (1′-H), 3.91 (1″-H), 3.65 (3-H), 3.73 (5-H), 3.53 (5′-H), 3.23 (2′-H), 3.14 (6-OCH₃), 2.61 (2-H), 2.48 (3′-H), 2.25 (3′-N(CH₃)₂,), 1.82 (4-H), 1.91 (14a-H), 2.03 (8-H), 1.66 (4′a-H), 1.49 (14b-H), 1.35 (18-CH₃), 1.28 (16-CH₃), 1.25 (20-CH₃), 1.25 (5′-CH₃), 1.25 (4′b-H), 1.15 (1″-(CH₃)₂), 1.14 (21-CH₃), 1.05 (17-CH₃), 0.96 (19-CH₃), 0.88 (15-CH₃).

¹³C NMR (75 MHz, CDCl₃) [δ/ppm] 175.8 (1-C), 155.1 (9a-NCONH), 106.6 (1′-C), 90.2 (5-C), 79.5 (6-C), 78.9 (3-C), 74.2 (11-C), 74.7 (12-C), 70.5 (2′-C), 70.0 (5′-C), 65.7 (3′-C), 49.8 (6-OCH₃), 44.8 (2-C), 42.7 (1″-C), 40.3 (3′N—(CH₃)₂), 36.4 (4-C), 28.1 (4′-C), 27.5 (8-C), 23.6 (18-CH₃), 23.3 (1″-(CH₃)₂), 22.3 (14-C), 21.3 (5′-CH₃), 20.6 (19-CH₃), 16.9 (21-CH₃), 15.5 (16-CH₃), 12.6 (20-CH₃), 11.2 (15-CH₃), 7.7 (17-CH₃).

### EXAMPLE 22

### 2′-O-Acetyl-3-decladinosyl-6-O-Me-9-deoxo-9-dihydro-9a-N-(N′-isopropylcarbamoyl)-9a-aza-9a-homoerythromycin A

To a solution of 3-decladinosyl-6-O-Me-9-deoxo-9-dihydro-9a-N-(N′-isopropylcarbamoyl)-9a-aza-9a-homoerythromycin A (1.08 g) in CH₂Cl₂ (20 ml), NaHCO₃ (0.54 g) and acetic acid anhydride (166 μl) were added and it was then stirred for 20 hours at room temperature. Onto the reaction mixture a saturated NaHCO₃ solution was added, the layers were separated and the aqueous one was extracted two more times with CH₂Cl₂. The combined organic extracts were rinsed with saturated NaHCO₃ solution and water and evaporated yielding the title product (0.68 g) with following physical-chemical constants:

TLC R_{f}=0.57 (CHCl₃: MeOH:NH₄OH=6:1:0.1).

IR (KBr) [νcm⁻¹]: 3431, 2974, 2937, 2876, 1734, 1629, 1524, 1459, 1376, 1243, 1167, 1083, 1058, 985, 957, 938, 904, 806, 671.

MS m/z: (ES): MH⁺=718.22

### EXAMPLE 23

### 2′-O-Acetyl-3-O-mesyl-6-O-Me-3-decladinosyl-9-deoxo-9-dihydro-9a-N—(N′-isopropylcarbamoyl)-9a-aza-9a-homoerythromycin A

To a solution of 2′-O-Acetyl-3-decladinosyl-6-O-Me-9-deoxo-9-dihydro-9a-N—(N′-isopropylcarbamoyl)-9a-aza-9a-homoerythromycin A (0.35 g) in pyridine (15 ml) methylsulphonyl anhydride (0.30 g) was added and the reaction mixture was stirred at room temperature for 3 hours. The solvent was evaporated and the residue was dissolved in CH₂Cl₂ (20 ml). Saturated aqueous solution of NaHCO₃ (20 ml) was added, the layers were separated and the water layer was extracted two more times with CH₂Cl₂ Combined organic extracts were rinsed with NaHCO₃ and brine, dried over K₂CO₃ and evaporated yielding 0.36 g of crude product with following physical-chemical constants

TLC R_{f}0.76 (CHCl₃:MeOH:NH₄OH=6:1:0.1).

IR (KBr) [νcm⁻¹]: 3427, 2975, 2932, 2877, 2854, 1739, 1628, 1524, 1462, 1375, 1342, 1243, 1175, 1112, 1060, 958, 917, 830, 768, 707, 669.

MS m/z: (ES): MH⁺=796.28

### EXAMPLE 24

### 3-keto-3-decladinosyl-6-O-Me-9-deoxo-9-dihydro-9a-N-(N-isopropylcarbamoyl)-9a-aza-9a-homoerythromycin A

To a solution of 2′-O-Acetyl-3-decladinosyl-6-O-Me-9-deoxo-9-dihydro-9a-N-(N′-isopropylcarbamoyl)-9a-aza-9a-homoerythromycin A (0.18 g) in CH₂Cl₂ (10 ml) dimethylsulfoxide (0.21 ml) and N,N-dimethyl-aminopropyl-ethyl-carbodiimide (0.28 ml) were added. The reaction mixture was cooled to 15° C., a then, keeping the temperature constant, solution of pyridinium trifluoracetate (0.22 g) in CH₂Cl₂ (5 ml) was added drop wise during 30 minutes. The reaction mixture was stirred at 15° C. to room temperature for additional 2 hours. To the reaction mixture saturated aqueous solution of NaCl (20 ml) was added and the pH value was adjusted to pH 9.5. The layers were separated and the water layer was extracted two more times with CH₂Cl₂ Combined organic extracts were rinsed with brine, NaHCO₃ and water, dried over K₂CO₃ and evaporated yielding 0.15 g of the crude product which is dissolved in MeOH (20 ml) and stirred for 24 hours at room temperature. The solvent was evaporated and the residue purified by low pressure chromatography on a silica gel column using the system CH₂Cl₂:MeOH:NH₄OH=90:3:0.3. The combining and evaporating of chromatographically homogenous fractions gave the title product (0.074 g) with following physical-chemical constants:

TLC R_{f}0.32 (CH₂Cl₂:MeOH:NH₄OH=90:9:0.5).

IR (KBr) [νcm⁻¹]: 3428, 2936, 1741, 1629, 1520, 1458, 1378, 1260, 1172, 1111, 1077, 1050, 985, 810.

MS m/z: (ES): MH⁺=674.27

¹H NMR (300 MHz, CDCl₃) [δ/ppm] 5.11 (13-H), 4.42 (1′-H), 4.34 (5-H), 3.91 (1″-H), 3.82 (2-H), 3.63 (5′-H), 3.21 (2′-H), 3.12 (4-H), 2.99 (6-OCH₃), 2.57 (3′-H), 2.33 (3′-N(CH₃)₂,), 2.04 (8-H), 1.94 (14a-H), 1.75 (4′a-H), 1.54 (14b-H), 1.38 (18-CH₃), 1.33 (16-CH₃), 1.26 (20-CH₃), 1.27 (5′-CH₃), 1.27 (4′b-H), 1.16 (1″-(CH₃)₂), 1.14 (21-CH₃), 1.27 (17-CH₃), 0.98 (19-CH₃), 0.90 (15-CH₃).

¹³C NMR (75 MHz, CDCl₃) [δ/ppm] 207.0 (3-C), 170.6 (1-C), 158.0 (9a-NCONH), 102.8 (1′-C), 75.8 (5-C), 79.4 (6-C), 74.4 (12-C), 74.2 (11-C), 70.3 (2′-C), 69.2 (5′-C), 65.8 (3′-C), 50.7 (2-C), 50.1 (6-OCH₃), 45.7 (4-C), 42.8 (1″-C), 40.4 (3′N—(CH₃)₂), 29.1 (4′-C), 27.4 (8-C), 23.8 (18-CH₃), 23.2 (1″-(CH₃)₂), 22.4 (14-C), 21.2 (5′-CH₃), 20.8 (19-CH₃), 16.8 (21-CH₃), 14.0 (16-CH₃), 13.2 (20-CH₃), 12.5 (17-CH₃), 11.3 (15-CH₃).

### EXAMPLE 25

### 2,3-anhydro-3-decladinosyl-6-O-Me-9-deoxo-9-dihydro-9a-N—(N′-isopropylcarbamoyl)-9a-aza-9a-homoerythromycin A

To a solution of 2′-O-Acetyl-3-O-mesyl-3-decladinosyl-6-O-Me-9-deoxo-9-dihydro-9a-N-(N′-isopropylcarbamoyl)-9a-aza-9a-homoerythromycin A (0.35 g) in DMF/THF (17 ml/5 ml) suspension (60%) of NaH in mineral oil (90 mg) was added and the reaction mixture was stirred at 0° C. for 5 hours. The reaction mixture was poured into saturated aqueos solution of NaHCO₃ (20 ml), EtOAc (20 ml) was added and the layers were separated. The water layer was extracted two more times with EtOAc. Combined organic extracts were rinsed with NaHCO₃ and brine, dried over K₂CO₃ and evaporated yielding 0.53 g of product. The obtained product was dissolved in MeOH (20 ml) was stirred for 24 hours at room temperature. The solvent was evaporated and the crude product was purified by chromatography on a silica gel column using the system (CH₂Cl₂:MeOH:NH₄OH=90:3:0.3). The combining and evaporating of chromatographically homogenous fractions gave the title product (0.031 g) with following physical-chemical constants:

TLC R_{f}=0.50 (CHCl₃:MeOH:NH₄OH=6:1:0.1).

IR (KBr) [νcm⁻¹]: 3439, 2974, 2937, 1735, 1628, 1520, 1459, 1381, 1336, 1270, 1175, 1111, 1075, 1051, 958, 919, 832, 766, 535.

MS m/z: (ES): MH⁺=658.22

¹H NMR (300 MHz, CDCl₃) [δ/ppm] 5.16 (13-H), 4.93 (3-H), 4.56 (1′-H), 3.90 (1″-H), 3.23 (5-H), 3.53 (5′-H), 3.27 (2′-H), 3.12 (6-OCH₃), 2.70 (3′-H), 2.36 (3′-N(CH₃)₂,), 1.52 (4-H), 1.91 (14a-H), 2.05 (8-H), 1.70 (4′a-H), 1.49 (14b-H), 1.36 (18-CH₃), 1.30 (16-CH₃), 1.25 (20-CH₃), 1.25 (5′-CH₃), 1.25 (4′b-H), 1.15 (1″-(CH₃)₂), 1.12 (21-CH₃), 1.06 (17-CH₃), 0.96 (19-CH₃), 0.88 (15-CH₃).

¹³C NMR (75 MHz, CDCl₃) [δ/ppm] 173.2 (1-C), 158.1 (9a-NCONH), 146.4 (2-C), 127.4 (3-C), 106.7 (1′-C), 86.1 (5-C), 79.5 (6-C), 74.2 (11-C), 75.0 (12-C), 70.7 (2′-C), 68.3 (5′-C), 65.9 (3′-C), 50.7 (6-OCH₃), 42.8 (1″-C), 39.0 (3′N—(CH₃)₂),40.4 (4-C), 29.1 (4′-C), 28.4 (8-C), 23.4 (18-CH₃), 22.3 (14-C), 22.1 (1″-(CH₃)₂), 20.9 (5′-CH₃), 20.5 (19-CH₃), 16.8 (21-CH₃), 14.2 (16-CH₃), 12.5 (20-CH₃), 11.4 (15-CH₃), 8.8 (17-CH₃).

### EXAMPLE 26

### 3-Decladinosyl-6-O-Me-9-deoxo-9-dihydro-9a-N—[N′-(2,4-dichlorophenyl)carbamoyl]-9a-aza-9a-homoerythromycin A

To a solution of 3-decladinosyl-6-O-Me-9-deoxo-9-dihydro-9a-aza-9a-homoerythromycin A (0.20 g) in acetonitrile (20 ml) 2,4-dichlorophenylisocyanate (0.064 6) was added. Reaction mixture was stirred for 30 min at room temperature. The solvent was evaporated and 0.254 g of crude product is obtained. Crude product was purified by chromatography on a silica gel column using the system (CH₂Cl₂:MeOH:NH₄OH=90:5:0.5). The combining and evaporating of chromatographically homogenous fractions gave the title product (0.169 g) with following physical-chemical constants:

TLC R_{f}=0.61 (CHCl₃:MeOH:NH₄OH=6:1:0.1).

IR (KBr) [νcm⁻¹]: 3448, 2975, 2939, 2879, 2787, 1729, 1707, 1670, 1582, 1517, 1459, 1382, 1328, 1298, 1274, 1165, 1112, 1076, 1051, 983, 956, 937, 897, 861, 822, 750, 699.

MS m/z: (ES): MH⁺=778.6

¹H NMR (300 MHz, CDCl₃) [δ/ppm] 8.13 (6″-H), 7.34 (3″-H), 7.18 (5″-H), 5.11 (13-H), 4.39 (1′-H), 3.81 (5-H), 3.72 (3-H), 3.52 (5′-H), 3.35 (6-OCH₃), 3.18 (2′-H), 2.61 (2-H), 2.61 (10-H), 2.50 (3′-H), 2.27 (3′-N(CH₃)₂), 2.04 (8-H), 1.93 (14a-H), 1.89 (4-H), 1.68 (4′a-H), 1.52 (14b-H), 1.35 (6-CH₃), 1.33 (4′b-H), 1.28 (2-CH₃), 1.25 (10-CH₃), 1.23 (5′-CH₃), 1.18 (12-CH₃), 1.04 (4-CH₃), 1.02 (8-CH₃), 0.89 (15-CH₃).

¹³C NMR (75 MHz, CDCl₃) [δ/ppm] 176.3 (1-C), 155.0 (9a-NCONH), 134.9 (1″-C), 128.4 (3″-C), 127.7 (5″-C), 127.1 (2″-C), 122.7 (4″-C), 122.1 (6″-C), 106.9 (1′-C), 90.3 (5-C), 79.7 (3-C), 79.1 (6-C), 76.1 (13-C), 73.6 (11-C), 73.6 (12-C), 70.5 (2′-C), 69.9 (5′-C), 65.7 (3′-C), 49.9 (6-O—CH₃), 44.7 (2-C), 40.3 (3′N—(CH₃)₂), 36.7 (4-C), 35.5 (7-C), 28.3 (4′-H), 28.2 (8-C), 22.4 (14-C), 21.3 (5′-CH₃), 20.6 (18-CH₃), 16.9 (21-CH₃), 15.4 (16-CH₃), 20.5 (20-CH₃), 11.4 (15-CH₃), 7.6 (17-CH₃).

### EXAMPLE 27

### 2′-O-Acetyl-3-decladinosyl-6-O-Me-9-deoxo-9-dihydro-9a-N—[N′-(2,4-dichlorophenyl)carbamoyl]-9a-aza-9a-homoerythromycin A

To a solution of 3-decladinosyl-6-O-Me-9-deoxo-9-dihydro-9a-N—[N′-(2,4-dichlorophenyl)carbamoyl]-9a-aza-9a-homoerythromycin A (0.12 g) in CH₂Cl₂ (10 ml), NaHCO₃ (0.058 g) and acetic acid anhydride (15 μl) were added and it was then stirred for 24 hours at room temperature. Onto the reaction mixture a saturated NaHCO₃ solution was added, the layers were separated and the aqueous one was extracted two more times with CH₂Cl₂. The combined organic extracts were rinsed with saturated NaHCO₃ solution and water and evaporated yielding the title product (0.115 g) with following physical-chemical constants:

TLC R_{f}=0.29 (CHCl₃:MeOH:NH₄OH=90:9:0.5).

IR (KBr) [νcm⁻¹]: 3449, 2973, 2939, 1748, 1730, 1668, 1582, 1517, 1459, 1377, 1298, 1261, 1239, 1165, 1098, 1050, 985, 906, 866, 809, 764, 664, 622, 584, 544, 505, 483, 463, 444, 386.

MS m/z: (ES): MH⁺=820.19

### EXAMPLE 28

### 2′-O-Acetyl-3-O-mesyl-6-O-Me-3-decladinosyl-9-deoxo-9-dihydro-9a-N—[N′-(2,4-dichlorophenyl)carbamoyl]-9a-aza-9a-homoerythromycin A

To a solution of 2′-O-Acetyl-3-decladinosyl-6-O-Me-9-deoxo-9-dihydro-9a-N—[N-(2,4-dichlorophenyl)carbamoyl]-9a-aza-9a-homoerythromycin A (1.50 g) in pyridine (60 ml) methylsulphonyl anhydride (1.16 g) was added and the reaction mixture was stirred at room temperature for 3 hours. The solvent was evaporated and the residue was dissolved in CH₂Cl₂ (50 ml). Saturated aqueous solution of NaHCO₃ (50 ml) was added, the layers were separated and the water layer was extracted two more times with CH₂Cl₂ Combined organic extracts were rinsed with NaHCO₃ and brine, dried over K₂CO₃ and evaporated yielding 1.57 g of crude product with following physical-chemical constants

TLC R_{f}=0.68 (etihylacetate:hexane:diethylamine=10:10:2).

IR (KBr) [νcm⁻¹]: 3434, 3060, 2974, 2935, 1736, 1701, 1686, 1655, 1637, 1560, 1528, 1509, 1486, 1376, 1330, 1240, 1208, 1193, 1059, 785, 773, 753, 681, 609.

MS m/z: (ES): MH⁺=898.09

### EXAMPLE 29

### 3-keto-3-decladinosyl-6-O-Me-9-deoxo-9-dihydro-9a-N-[N′-(2,4-dichlorophenyl)carbamoyl]-9a-aza-9a-homoerythromycin A

To a solution of 2′-O-Acetyl-3-decladinosyl-6-O-Me-9-deoxo-9-dihydro-9a-N-[N′(2,4-dichlorophenyl)carbamoyl]-9a-aza-9a-homoerythromycin A (0.73 g) in CH₂Cl₂ (20 ml) dimethylsulfoxide (1.00 ml) and N,N-dimethyl-aminopropyl-ethyl-carbodiimide (1.02 6) were added. The reaction mixture was cooled to 15° C., a then, keeping the temperature constant, solution of pyridinium trifluoracetate (1.03 g) in CH₂Cl₂ (20 ml) was added drop wise during 30 minutes. The reaction mixture was stirred at 15° C. to room temperature for additional 10 hours. To the reaction mixture saturated aqueous solution of NaCl (20 ml) was added and the pH value was adjusted to pH 9.5. The layers were separated and the water layer was extracted two more times with CH₂Cl₂ Combined organic extracts were rinsed with brine, NaHCO₃ and water, dried over K₂CO₃ and evaporated yielding 0.63 g of the crude product which is dissolved in MeOH (20 ml) and stirred for 24 hours at room temperature. The solvent was evaporated and the residue purified by low pressure chromatography on a silica gel column using the system (CH₂Cl₂:MeOH:NH₄OH=90:5:0.5. The combining and evaporating of chromatographically homogenous fractions gave the title product (0.253 g) with following physical-chemical constants:

TLC R_{f}=0.61 (CHCl₃:MeOH:NH₄OH=6:1:0.1).

IR (KBr) [νcm⁻¹]: 3448, 2976, 2938, 2877, 2786, 1741, 1719, 1665, 1580, 1515, 1459, 1381, 1298, 1230, 1197, 1167, 1147, 1110, 1076, 1051, 986, 957, 937, 895, 859, 821, 761, 753, 622.

MS m/z: (ES): MH⁺=776.6

¹H NMR (300 MHz, CDCl₃) [δ/ppm] 8.10 (6″-H), 7.34 (3″-H), 7.18 (5″-H), 5.08 (13-H), 4.40 (1′-H), 4.31 (5-H), 3.81 (2-H), 3.62 (5′-H), 3.44 (6-OCH₃), 3.17 (4-H), 3.16 (2′-H), 2.75 (10-H), 2.49 (3′-H), 2.28 (3′-N(CH₃)₂), 2.19 (8-H), 1.94 (14a-H), 1.68 (4′a-H), 1.56 (14b-H), 1.32 (6-CH₃), 1.30 (2-CH₃), 1.27 (7a-H), 1.24 (10-CH₃), 1.23 (4′b-H), 1.23 (5′-CH₃), 1.19 (12-CH₃), 1.06 (4-CH₃), 1.03 (7b-H), 1.02 (8-CH₃), 0.91 (15-CH₃).

¹³C NMR (75 MHz, CDCl₃) [δ/ppm] 207.1 (3-C), 171.1 (1-C), 155.6 (9a-NCONH), 135.1 (1″-C), 128.8 (3″-C), 128.2 (5″-C), 127.8 (2″-C), 123.1 (4″-C), 122.5 (6″-C), 103.1 (1′-C), 79.9 (6-C), 79.5 (13-C), 76.2 (5-C), 73.9 (12-C), 73.4 (11-C), 70.8 (2′-C), 69.7 (5′-C), 65.9 (3′-C), 51.1 (2-C), 50.9 (4-C), 50.6 (6-O—CH₃), 40.8 (3′N—(CH₃)₂), 39.0 (7-C), 29.3 (4′-H), 28.6 (8-C), 22.7 (14-C), 21.6 (5′-CH₃), 21.3 (19-CH₃), 17.2 (21-CH₃), 13.6 (16-CH₃), 12.8 (20-CH₃), 11.6 (15-CH₃), 7.6 (17-CH₃).

### EXAMPLE 30

### 2,3-anhydro-3-decladinosyl-6-O-Me-9-deoxo-9-dihydro-9a-N—[N′-(2,4-dichlorophenyl)carbamoyl]-9a-aza-9a-homoerythromycin A

To a solution of 2′-O-Acetyl-3-O-mesyl-3-decladinosyl-6-O-Me-9-deoxo-9-dihydro-9a-N-[N′-(2,4-dichlorophenyl)carbamoyl]-9a-aza-9a-homoerythromycin A (1.00 g) in DMF/THF (45 ml/13 ml) suspension (60%) of NaH in mineral oil (400 mg) was added and the reaction mixture was stirred at 0° C. The reaction mixture was poured into saturated aqueos solution of NaHCO₃ (50 ml), EtOAc (50 ml) was added and the layers were separated. The water layer was extracted two more times with EtOAc. Combined organic extracts were rinsed with NaHCO₃ and brine, dried over K₂CO₃ and evaporated yielding 0.32 g of product. The obtained product was dissolved in MeOH (20 ml) was stirred for 24 hours at room temperature. The solvent was evaporated and the crude product was purified by chromatography on a silica gel column using the system (CH₂Cl₂:MeOH:NH₄OH=90:3:0.3). The combining and evaporating of chromatographically homogenous fractions gave the title product (0.024 g) with following physical-chemical constants:

TLC R_{f}=0.43 (etihylacetate:hexane:diethylamine=10:10:2).

IR (KBr) [νcm⁻¹]: 3452, 2981, 2941, 1748, 1731, 1662, 1582, 1516, 1459, 137, 1298, 1261, 1243, 1165, 1098, 1052, 985, 906, 867, 810, 764, 666, 625, 587.

MS m/z: (ES): MH⁺=760.70

¹H NMR (300 MHz, CDCl₃) [δ/ppm] 8.12 (6″-H), 7.34 (3″-H), 7.18 (5″-H), 5.16 (13-H), 4.92 (3-H), 4.39 (1′-H), 3.20 (5-H), 3.52 (5′-H), 3.30 (6-OCH₃), 3.18 (2′-H), 2.61 (10-H), 2.50 (3′-H), 2.27 (3′-N(CH₃)₂), 2.04 (8-H), 1.93 (14a-H), 1.68 (4′a-H), 1.55 (4-H), 1.52 (14b-H), 1.35 (6-CH₃), 1.29 (2-CH₃), 1.26 (4′b-H), 1.25 (10-CH₃), 1.25 (5′-CH₃), 1.13 (12-CH₃), 1.04 (4-CH₃), 0.97 (8-CH₃), 0.89 (15-CH₃).

¹³C NMR (75 MHz, CDCl₃) [δ/ppm] 176.3 (1-C), 156.0 (9a-NCONH), 146.8 (2-C), 127.7 (3-C), 134.8 (1″-C), 128.3 (3″-C), 127.5 (5″-C), 127.0 (2″-C), 122.8 (4″-C), 122.1 (6″-C), 106.9 (1′-C), 86.3 (5-C), 79.1 (6-C), 76.1 (13-C), 73.4 (11-C), 73.5 (12-C), 70.7 (2′-C), 69.9 (5′-C), 65.7 (3′-C), 50.0 (6-O—CH₃), 40.3 (3′N—(CH₃)₂), 40.7 (4-C), 35.4 (7-C), 28.3 (4′-H), 28.2 (8-C), 22.4 (14-C), 21.3 (5′-CH₃), 20.6 (8-CH₃), 16.9 (21-CH₃), 14.2 (2-CH₃), 12.8 (20-CH₃); 11 v4 (15-CH₃), 7.6 (4-CH₃).

1. Novel 3-decladinosyl derivatives of 9a-N-carbamoyl- and 9a-N-thiocarbamoyl-9-deoxo-9-dihydro-9a-aza-9a-homoerythromycin A of the general formula (I), 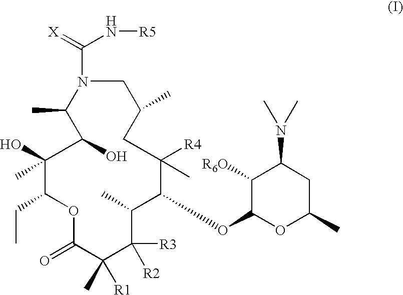
their pharmaceutically acceptable addition salts with inorganic or organic acids and their hydrates,
wherein
R₁ individually stands for hydrogen or together with R₂ stands for double bond,
R₂ individually stands for hydrogen, hydroxyl or a group of the formula (II), 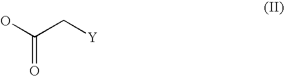
wherein
Y individually stands for monocyclic aromatic ring, unsubstituted or substituted with groups which are selected independently from halogen, OH, OMe, NO₂, NH₂
or
R₂ together with R₃ stands for ketone or together with R₁ stands for double bond,
R₃ individually stands for hydrogen or together with R₂ stands for ketone or together with R₄ stands for ether,
R₄ individually stands for hydroxyl, OCH₃ group or together with R₃ stands for ether,
R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, and m is 0-3,
R₆ individually stands for hydrogen or hydroxyl protected group, and
X stands for oxygen or sulphur.

2. Compound according to claim 1. characterized in that R₁ and R₃ stands for hydrogen, R₂ stands for hydroxyl, R₄ individually stands for hydroxyl or for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stands for hydrogen.

3. Compound according to claim 2, characterized in that R₄ stands for hydroxyl, R₅ stands for ethyl group and X stands for oxygen.

4. Compound according to claim 2, characterized in that R₄ stands for hydroxyl, R₅ stands for t-butyl group and X stands for oxygen.

5. Compound according to claim 2, characterized in that R₄ stands for hydroxyl, R₅ stands for isopropyl group and X stands for oxygen.

6. Compound according to claim 2, characterized in that R₄ stands for OCH₃ group, R₅ stands for isopropyl group and X stands for oxygen.

7. Compound according to claim 2, characterized in that R₄ stands for hydroxyl, R₅ stands for allyl group and X stands for sulphur.

8. Compound according to claim 2, characterized in that R₄ stands for hydroxyl, R₅ stands for benzyl group and X stands for oxygen.

9. Compound according to claim 2, characterized in that R₄ stands for hydroxyl, R₅ stands for benzyl group and X stands for sulphur.

10. Compound according to claim 2, characterized in that R₄ stands for hydroxyl, R₅ stands for 2-trifluormethylphenyl group and X stands for oxygen.

11. Compound according to claim 2, characterized in that R₄ stands for hydroxyl, R₅ stands for 3-trifluormethylphenyl group and X stands for oxygen.

12. Compound according to claim 2, characterized in that R₄ stands for hydroxyl, R₅ stands for 3-trifluormethylphenyl group and X stands for sulphur.

13. Compound according to claim 2, characterized in that R₄ stands for OCH₃ group, R₅ stands for 2,4-dichlorophenyl group and X stands for oxygen.

14. Compound according to claim 2, characterized in that R₄ stands for hydroxyl, R₅ stands for 2,4-dichlorophenyl group and X stands for oxygen.

15. Compound according to claim 1, characterized in that R₁ and R₃ stands for hydrogen, R₂ stands for a group of the formula (II), wherein Y individually stands for monocyclic aromatic ring, unsubstituted or substituted with groups which are selected independently from halogen, OH, OMe, NO₂, NH₂, R₄ individually stands for hydroxyl or for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stands for hydrogen.

16. Compound according to claim 15, characterized in that Y stands for 4-NO₂ substituted phenyl, R₄ stands for hydroxyl, R₅ stands for isopropyl group and X stands for oxygen.

17. Compound according to claim 15, characterized in that Y stands for 4-NO₂ substituted phenyl, R₄ stands for hydroxyl, R₅ stands for 2,4-dichlorophenyl group and X stands for oxygen.

18. Compound according to claim 1, characterized in that R₁, R₂ and R₆ stands for hydrogen, R₃ together with R₄ stands for ether, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur.

19. Compound according to claim 18, characterized in that R₅ stands for isopropyl group and X stands for oxygen.

20. Compound according to claim 18, characterized in that R₅ stands for 2,4-dichlorophenyl group and X stands for oxygen.

21. Compound according to claim 1, characterized in that R₁ and R₆ stands for hydrogen, R₂ stands for hydroxyl, R₃ together with R₄ stands for ether, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3 and X stands for oxygen or sulphur.

22. Compound according to claim 21, characterized in that R₅ stands for isopropyl group and X stands for oxygen.

23. Compound according to claim 21, characterized in that R₅ stands for 2,4-dichlorophenyl group and X stands for oxygen.

24. Compound according to claim 1, characterized in that R₁ and R₆ stands for hydrogen, R₂ together with R₃ stands for ketone, R₄ stands for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3 and X stands for oxygen or sulphur.

25. Compound according to claim 24, characterized in that R₅ stands for isopropyl group and X stands for oxygen.

26. Compound according to claim 24, characterized in that R₅ stands for 2,4-dichlorophenyl group and X stands for oxygen.

27. Compound according to claim 1, characterized in that R₁ together with R₂ stands for double bond, R₃ and R₆ stands for hydrogen, R₄ stands for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3 and X stands for oxygen or sulphur.

28. Compound according to claim 27, characterized in that R₅ stands for isopropyl group and X stands for oxygen.

29. Compound according to claim 24, characterized in that R₅ stands for 2,4-dichlorophenyl group and X stands for oxygen.

30. A process for preparation of compounds of the formula (I), 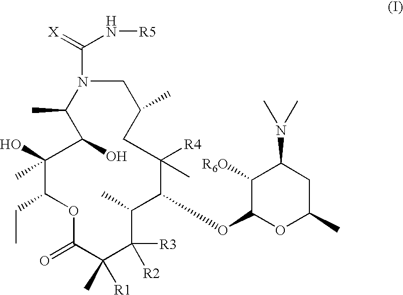
their pharmaceutically acceptable addition salts with inorganic or organic acids and their hydrates,
wherein
R₁ individually stands for hydrogen or together with R₂ stands for double bond,
R₂ individually stands for hydrogen, hydroxyl or a group of the formula (II), 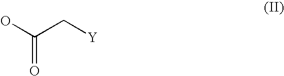
wherein
Y individually stands for monocyclic aromatic ring, unsubstituted or substituted with groups which are selected independently from halogen, OH, OMe, NO₂, NH₂
or
R₂ together with R₃ stands for ketone or together with R₁ stands for double bond,
R₃ individually stands for hydrogen or together with R₂ stands for ketone or together with R₄ stands for ether,
R₄ individually stands for hydroxyl, OCH₃ group or together with R₃ stands for ether,
R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl and m is 0-3,
R₆ individually stands for hydrogen or hydroxyl protected group,
X stands for oxygen or sulphur,
Comprising the steps:
a) subjecting starting compounds of the formula 1 (scheme 1) wherein R₄ individually stands for hydroxyl or OCH₃ group to a reaction with isocyanate or isothiocyanate of the formula R₅—N═C═X wherein R₅ and X have the above meanings, yielding compounds of general formula (I), wherein R₁ and R₃ stands for hydrogen, R₂ stands for hydroxyl, R₄ individually stands for hydroxyl or for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stands for hydrogen; and
b) selectively acylating the hydroxyl group at 2′-position, in the presence of inorganic or organic base yielding 2′-O-acyl derivatives of the general formula (I), wherein R₆ stands for acetyl group and substituents R₁, R₂, R₃, R₄, R₅ and X have the meanings defined in a);
c1) optionally reacting a mixed anhydride of carboxylic acids of the formula Z-COO—R′, wherein Z individually stands for hydrogen or for group Y, which is defined above, R′ stands for a protecting group, in the presence of inorganic or organic base, yielding compounds of the general formula (I), wherein R₁ and R₃ stands for hydrogen, R₂ stands for a group of the formula (II), wherein Y individually stands for monocyclic aromatic ring, unsubstituted or substituted with groups which are selected independently from halogen, OH, OMe, NO₂, NH₂, R₄ individually stands for hydroxyl or for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stands for acetyl group then deprotecting with a lower alcohol, yielding a compound of the formula (I), wherein R₆ stands for hydrogen and all other substituents have the above meanings,
c2) or, when R₄ stands for OCH₃ group and the remaining substituents have the meanings defined in b), optionally oxidizing the hydroxyl group in the C-3 position of an aglycone ring with N,N-dimethylaminopropyl-3-ethyl-carbodiimide, yielding compounds of the general formula (I), wherein R₁ stand for hydrogen, R₂ together with R₃ stands for ketone, R₄ stands for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stand for acetyl, then deprotecting with a lower alcohol, yielding a compound of the formula (I), wherein R₆ stands for hydrogen and all other substituents have the above meanings,
c3) or, when R₄ stands for hydroxyl and the remaining substituents have the meanings defined in b), optionally oxidizing to obtain compounds of the general formula (I) from the step c2), yield compounds with 3,6-hemiketal structure given by general formula (I), wherein R₁ stand for hydrogen, R₂ stands for hydroxyl, R₃ together with R₄ stands for ether, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stand for acetyl, then deprotecting with a lower alcohol, yielding a compound of the formula (I), wherein R₆ stands for hydrogen and all other substituents have the above meanings,
c4) or when R₄ stands for OCH₃ group and the remaining substituents have the meanings defined in b), optionally dehydrating to transform hydroxyl group on position 3, then subjecting to an elimination reaction, yielding 2,3-anhydro derivatives of the general formula (I), wherein R₁ together with R₂ stands for double bond, R₃ stands for hydrogen, R₄ stands for OCH₃ group, R₅ individually stands for C₁-C₄ alkyl group, C₂-C₄ alkenyl group, —(CH₂)ₘ—Ar, wherein Ar individually stands for phenyl or phenyl substituted with one or two groups which are selected independently from halogen or halogen alkyl, m is 0-3, X stands for oxygen or sulphur and R₆ stands for acetyl then deprotecting with a lower alcohol, yielding a compound of the general formula (I), wherein R₆ stands for hydrogen and all other substituents have the above meanings
c5) or, when R₄ stands for hydroxyl and the remaining substituents have the meanings defined in b), optionally eliminating, yielding 3,6-cyclic ether of the general formula (I), wherein R₁ and R₂ stands for hydrogen, R₃ together with R₄ stands for ether, R₅ individually stands for C₁-C₄ alkyl group, C₂-C₄ alkenyl group, —(CH₂)ₘ, Ar, wherein Ar individually stands for phenyl or phenyl substituted with one or two groups which are selected independently from halogen or halogen alkyl, m is 0-3, X stands for oxygen or sulphur and R₆ stands for acetyl then deprotecting with a lower alcohol, yielding a compound of the general formula (I), wherein R₆ stands for hydrogen and all other substituents have the above meanings.
